(19) United States
(12) Patent Application Publication
(10) Pub. No.: US 2017/0051279 A1
(43) Pub. Date: Feb. 23, 2017

(54) VASCULAR RE-MODELLING

(71) Applicant: Academisch Ziekenhuis Leiden a/u Leiden University Medical Center, Leiden (NL)

(72) Inventors: Anne Yael Nossent, Den Haag (NL); Paulus Hubertus Andreas Quax, Voorschoten (NL); Antonius Johannes Nicolaas Bastiaansen, Leiden (NL); Sabine Marlies Janine Welten, Den Haag (NL); Anouk Wezel, Den Haag (NL); Ilze Bot, Papendrecht (NL)

(21) Appl. No.: 15/099,027

(22) Filed: Apr. 14, 2016

Related U.S. Application Data
(63) Continuation of application No. PCT/EP2014/072464, filed on Oct. 20, 2014.

(30) Foreign Application Priority Data
Oct. 18, 2013 (GB) 1318492.4

(51) Int. Cl. C12N 15/113

(52) U.S. Cl. CPC C12N 15/113; C12N 2310/113; C12N 2310/315; C12N 2310/321

(57) ABSTRACT

The present invention is based on the finding that microRNA from the microRNA gene cluster located on the human chromosomal at locus 14q32 play an important role in vascular development and re-modelling. Modulators of any of the 14q32 microRNA may be exploited as a means to modulate vascular re-modelling processes and/or in the treatment and/or prevention of vascular disorders or disease.

### RELATED APPLICATIONS

This application is a continuation of International Application No. PCT/EP2014/072464, which designated the United States and was filed on Oct. 20, 2014, published in English.

This application claims priority under 35 U.S.C. §119 or 365 to GB, Application No. 1318492.4, filed Oct. 18, 2013. The entire teachings of the above applications are incorporated herein by reference.

### FIELD OF THE INVENTION

This invention provides microRNAs inhibitor compounds for use in the treatment of vascular disorders and/or for modulating vascular re-modelling processes.

### BACKGROUND OF THE INVENTION

Cardiovascular disease is the leading cause of death in Europe and North America. Endovascular interventions like balloon angioplasty or bypass surgery can be life-saving in patients with severe occlusive arterial disease. In up to 50% of patients however, depending on the physiological location of the artery, intervention-induced restenosis leads to complete re-occlusion of the artery within one year.

Neovascularisation, angiogenesis and arteriogenesis, is the body's natural repair mechanism after ischemia. Therapeutic neovascularisation would restore blood flow to downstream tissues. Clinical trials aiming to stimulate neovascularisation however have been unsuccessful in the past.

An important problem with therapeutic neovascularisation is that factors that stimulate positive vascular remodelling like angiogenesis and arteriogenesis often also stimulate negative vascular remodelling, like atherosclerosis and restenosis. Known as the “Janus phenomenon” this causes obvious problems when stimulating neovascularisation in patients with occlusive arterial disease, particularly following an (endovascular) intervention.

Atherosclerosis is a complex, multifactorial disease in which various processes, including immune modulation and cholesterol homeostasis, are involved. Damage to the endothelial layer in large and medium-sized arteries results in local up-regulation of adhesion molecules and chemokine production, together facilitating the influx of monocytes into the vessel wall.ⁱ Subsequent uptake of oxidized lipids through scavenger receptors leads to the formation of early fatty streaks. Continued inflammation attracts multiple immune cells to the lesion, eventually resulting in an advanced atherosclerotic plaque.^{ii,iii} Advanced atherosclerotic plaques are defined as plaques with a large lipid core covered by a thin fibrous cap containing little collagen and few smooth muscle cells.^{iv,v} Rupture of an advanced plaque can cause severe acute cardiovascular events such as myocardial infarction and stroke. Despite current lipid lowering therapies, cardiovascular diseases are still the main cause of death in western society. Taking into account the multifactorial nature of atherosclerosis, improvement of treatment strategies may be accomplished by targeting the process as a whole rather than focusing on single factors.

MicroRNAs (miRs) are a class of short, non-coding RNAs, approximately 20 nucleotides long, capable of downregulating target gene expression at post-transcriptional level.^{vi} A single miR has on average 200 predicted target genes.^{vii} Their ability to fine-tune expression of multiple genes makes them excellent drug targets for complex diseases.

Inhibition of miRs in atherosclerosis has been investigated before in several studies. Rayner et al. showed that inhibiting miR-33 results in a lowering of plasma VLDL while increasing plasma HDL.^{viii} Besides regulation of cholesterol homeostasis, miRs have also been implicated in other cellular mechanisms affecting atherosclerosis. For example, miR-126 regulates post-transcriptional VCAM expression in response to triglyceride-rich lipoproteins.^{ix} Also, inhibition of MiR-92a has been demonstrated to up-regulate endothelial KLF-2 and KLF-4 expression,^{x} which are Kruppel-like Factors with atheroprotective properties.^{xi} Furthermore, smooth muscle cell proliferation and migration can be repressed by miR-195; consequently, neo-intima formation can be reduced by miR-195 gene therapy.^{xii} MiR-155 has been shown to repress the transcription factor Bcl6, thereby increasing NF-κB activation and CCL2 expression in macrophages.^{xiii} It seems therefore that results in this area of research are very promising. However, it is apparent that most of these studies focus on the effect of miRs on a single cell type or process, thereby doing injustice to the ability of miRs to exert a broad range of effects.

Yaël Nossent et at, 2013 (Annals of Surgery Vol. 258(5) 743-753 describe that the 14q32 MicroRNA-487b targets the anti-apoptotic insulin receptor substrate 1 in hypertension induced remodeling of the aorta.

### SUMMARY OF THE INVENTION

The present invention is based on the finding that microRNA from the microRNA gene cluster located on the human chromosomal at locus 14q32 (referred to hereinafter as the “14q32 microRNAs”) play an important role in vascular development and re-modelling. Thus the invention may be exploited as a means to modulate vascular re-modelling processes and/or in the treatment and/or prevention of vascular disorders or disease.

Thus in a first aspect, the invention provides modulators of one or more 14q32 microRNAs for modulating vascular re-modelling processes and/or for use in treating or preventing vascular disorders.

In a second aspect, the invention provides a method of modulating vascular re-modelling processes and/or for treating or preventing a vascular disorder, said method comprising administering modulators of one or more 14q32 microRNAs to a subject in need thereof. The modulators may be administered in a therapeutically effective amount. The subject in need thereof may be a human or animal subject.

It should be noted that throughout this specification the terms “comprise” and/or “comprising” are used to denote that embodiments of the invention “comprise” the noted features and as such, may also include other features. However, in the context of this invention, the terms “comprise” and “comprising” encompass embodiments in which the invention “consists essentially of” the relevant features or “consists of” the relevant features.

The term “vascular” may encompass any form of vessel within the human or animal body. Specifically, the term “vascular” may be applied to blood vessels such as arteries, capillaries and/or veins. Accordingly, the term “vascular re-modelling” may be applied to any alteration in the function, size, shape and/or structure of an artery, capillary and/or vein and/or the lumen they define. Thus a “vascular re-modelling process” may be defined as any process associated with, or leading to, alterations in vessel function, size, shape and/or structure. Similarly, the term “vascular disorder” may be applied to any disease, condition or syndrome affecting one or more types of blood vessel in the human or animal body.

Vascular re-modelling may take the form of positive re-modelling; that is, re-modelling comprising processes aimed at restoring vascular function such as, for example, neovascularisation, angiogenesis and/or arteriogenesis. Vascular re-modelling may also take the form of negative re-modelling; that is, re-modelling which leads to further vascular damage and/or adverse modulation of vascular structure and/or support. Negative re-modelling may comprise, for example, atherosclerosis and/or restenosis. Thus the term “vascular re-modelling” encompasses both positive and negative forms of re-modelling.

The term “vascular system” may encompass the vessels (arteries, capillaries and/or veins) within a particular tissue type, organ or region of the human or animal body. Thus the terms “vascular disorder” and “vascular re-modelling processes” may encompass diseases and processes affecting any of the vessels within a vascular system, for example the cardiovascular (coronary) systems as well as the vascular systems of the limbs (i.e. structures of the human or animal body such as legs and/or arms) as well as the pulmonary, cerebral, renal and/or hepatic organs, tissues and/or regions.

Accordingly the present invention may find application in the treatment and/or prevention of disorders, diseases, syndromes and/or conditions which affect any of the vessels and/or vascular systems of the human or animal body. Moreover, the invention may be exploited as a means to modulate vascular re-modelling processes in any of these vessels and/or systems.

Without wishing to be bound by theory, modulation of one or more 14q32 microRNAs, leads to the modulation of genes associated with vascular re-modelling. Positive re-modelling processes such as neovascularisation, angiogenesis and arteriogenesis form part of the body's natural response to vascular damage. However, it is known that negative re-modelling processes may occur simultaneously (or following) positive re-modelling processes (the Janus Phenomenon: see below). The inventors have discovered that inhibition of one or more of the 14q32 microRNAs not only offers an effective treatment for atherosclerosis and restenosis, it further stimulates positive re-modelling processes such as, for example neovascularisation, angiogenesis and arteriogenesis.

The term “modulate” as applied to “vascular re-modelling processes” may encompass any increase or decrease in the rate or occurrence/incidence of a vascular re-modelling process. Thus the 14q32 modulators disclosed in this invention may be exploited as a means to inhibit (prevent or suppress) and/or stimulate (encourage or increase) one or more vascular re-modelling processes. The 14q32 modulators and methods of this invention may be used to increase or stimulate re-modelling processes such as neovascularisation, angiogenesis and/or arteriogenesis. The degree of modulation affected by a 14q32 modulator of this invention may be assessed relative to “normal” or “control” levels of vascular re-modelling as might occur in cells or tissues not exhibiting pathology associated with vascular disease and/or not contacted with one or more of the 14q32 inhibitors described herein.

Specific examples of suitable modulators of 14q32 microRNAs expression are described elsewhere but the reader should note that a modulator of 14q32 microRNA expression is any molecule or compound capable of increasing and/or inhibiting (decreasing) the expression of the relevant microRNA. Typically, the modulators of this invention are microRNA inhibitors;

specifically, inhibitors of one or more of the 14q32 microRNAs. MicroRNA inhibitors may comprise compounds or molecules which inhibit or reduce the expression, function and/or activity of one or more microRNAs including the 14q32 microRNAs described herein. One of skill will appreciate that the degree of modulation may be assessed relative to a “normal” or “control” level of microRNA expression. For example, the degree of modulation may be assessed relative to the expression of the equivalent microRNA(s) in a test system (for example a cell based system) which does not exhibit pathology associated with vascular disease and/or which has not been subjected to, or contacted with, a 14q32 microRNA modulator.

The present invention may be applied to the treatment and/or prevention of peripheral artery disease and/or to the modulation of vascular re-modelling processes in peripheral arteries.

The invention may provide methods and/or modulators of one or more 14q32 microRNAs, for use in modulating vascular re-modelling processes in cardiovascular/coronary and/or cerebral vessels. Moreover, the invention may find application in the treatment and/or prevention of cardiovascular/coronary and/or cerebral artery diseases.

The term “cardiovascular” or “coronary” disease may embrace (severe) occlusive arterial disease, myocardial infarction (and/or vascular damage caused thereby), ischaemic stroke as well as tissue or vascular damage occurring as a consequence of an ischaemic event.

Additionally, the invention may be applied to the treatment and/or prevention (prophylaxis) of disorders, diseases, syndromes or conditions which may occur following disease, surgery or treatment. In the context of cardiovascular disease, the microRNA modulator compounds and methods of this invention may be used in the treatment and/or prevention of conditions such as restenosis and/or atherosclerosis.

The invention may also be applied to the modulation of vascular remodelling processes which might occur following, for example, mycocardial infarction (so called cardiac remodelling), aneurysm formation (including, but not limited to abdominal or thoracal aorta aneurysms). The invention may find application in the treatment and/or prevention of such conditions (or complications associated therewith).

Additionally, or alternatively, modulators of one or more 14q32 microRNAs may be used to treat or prevent restenosis as might occur following, for example, surgical procedures or interventions including, for example, bypass surgery (including coronary and peripheral bypass surgery, dialysis procedures (dialysis shunt remodelling) and/or the implantation of a stent or balloon-angioplasty techniques.

Additionally, the uses and methods of this invention may be applied to the treatment and/or prevention of disorders, diseases, conditions and/or syndromes which are associated with, or causative of, vascular disease and/or vascular re-modelling processes. For example, the inventors have noted that through modulation of one or more 14q32 microRNAs, it is possible to modulate blood cholesterol levels. One of skill will appreciate that elevated blood cholesterol levels are often associated with atherosclerosis and thus the invention may be applied to the treatment of atherosclerosis and other cholesterol related diseases, conditions or syndromes. The invention may be exploited as a means to control hypercholesterolemia.

The microRNA modulator compounds and methods of this invention may be used to modulate, for example increase, encourage, promote or enhance, neovascularisation, angiogenesis and/or arteriogenesis.

The inventors have noted that the methods and uses of this invention may overcome problems associated with the Janus phenomenon, a side effect often linked with prior art therapies for vascular disease. The Janus phenomenon states that factors that stimulate positive vascular remodelling (i.e. angiogenesis and arteriogenesis) also stimulate negative remodelling (i.e. atherosclerosis and restenosis). As stated, the inventors have discovered that through modulation of 14q32 microRNAs, it is possible to inhibit vascular diseases (such as atherosclerosis and the like), however, unlike other therapies which might also stimulate negative vascular re-modelling processes, the compounds, uses and methods of this invention (simultaneously) modulate, for example, increase, stimulate, enhance or promote neovascularisation, angiogenesis and/or arteriogenesis.

Specifically, while prior art treatments for vascular disease may stimulate neovascularisation, angiogenesis and/or arteriogenesis they can often also lead to restenosis and/or atherosclerosis. However, the inventors have observed that treatment of vascular disorders or diseases (as described herein) by inhibition of 14q32 microRNAs, leads to a decrease in atherosclerotic plaque formation and lesion size and an increase in positive re-modelling processes such as neovascularisation, arteriogenesis and/or angiogenesis.

In view of the above, the present invention provides modulators (for example inhibitors) of one or more 14q32 microRNAs for use in treating or preventing atherosclerosis. Indeed the inventors have discovered that modulation of one or more 14q32 microRNAs not only leads to a general decrease in atherosclerotic plaque formation and lesion size, but also to decreased necrotic core formation within the atherosclerotic plaque. Since necrotic core size correlates with plaque stability and plaque rupture, the microRNA modulators of this invention may be used to stabilise atherosclerotic plaques.

The invention further provides modulators of one or more 14q32 microRNAs for use in treating or preventing hypercholesterolimea or for modulating blood cholesterol levels.

The invention provides modulators of one or more 14q32 microRNAs for use in modulating plaque stability and/or for treating or preventing restenosis.

The invention further relates to modulators of one or more 14q32 microRNAs for use in modulating arteriogenesis and/or angiogenesis. It should be understood that one may further observe the simultaneous inhibition of atherosclerosis and/or restenosis.

For the avoidance of doubt, the invention further extends to methods of treating or preventing atherosclerosis, restenosis and/or hypercholesterolimea or for modulating angiogenesis, arteriogenesis and/or blood cholesterol levels, the methods comprising administering a therapeutically effective amount of a modulator of one or more 14q32 microRNAs to a subject in need thereof. Again, it should be understood that one advantage of the present invention is that medicaments of this invention (comprising modulators of 14q32 microRNAs) applied to the modulation of angiogenesis and/or arteriogenesis (namely positive vascular re-modelling) appear to cause significantly reduced levels of negative remodelling or disease, including, for example atherosclerosis and/or restenosis.

It should be understood that while this invention generally refers to “modulators” of 14q32 microRNAs, modulators which are inhibitors of 14q32 microRNAs may be of particular use.

One of skill will be familiar with the term “microRNA” (or “miRNA”). MicroRNAs are small non-coding RNA molecules of around 22 nucleotides in length which affect the regulation of gene expression. They are produced either from gene sequences or intron/exon sequences; many are encoded by intergenic sequences.

The human chromosomal locus, 14q32, encodes an array or cluster of microRNAs and each of these microRNAs is to be regarded as encompassed within the scope of this invention. Owing to their role in vascular remodeling, the 14q32 microRNAs may be collectively termed “vasoactive microRNAs”.

By way of example, the present invention may concern modulation, for example inhibition, of one or more microRNAs selected from the group consisting of:

1) microRNA-2392

2) microRNA-770

3) microRNA-493

4) microRNA-337

5) microRNA-665

6) microRNA-431

7) microRNA-433

8) microRNA-127

9) microRNA-432

10) microRNA-136

11) microRNA-370

12) microRNA-379

13) microRNA-411

14) microRNA-299

15) microRNA-380

16) microRNA-1197

17) microRNA-323a

18) microRNA-758

19) microRNA-329-1

20) microRNA-329-2

21) microRNA-494

22) microRNA-1193

23) microRNA-543

24) microRNA-495

25) microRNA-376c

26) microRNA-376a-2

27) microRNA-654

28) microRNA-376b

29) microRNA-376a-1

30) microRNA-300

31) microRNA-1185-1

32) microRNA-1185-2

33) microRNA-381

34) microRNA-487b

35) microRNA-539

36) microRNA-889

37) microRNA-544a

38) microRNA-655

39) microRNA-487a

40) microRNA-382

41) microRNA-134

42) microRNA-668

43) microRNA-485

44) microRNA-323b

45) microRNA-154

46) microRNA-496

47) microRNA-377

48) microRNA-541

49) microRNA-409

50) microRNA-412

51) microRNA-369

52) microRNA-410

53) microRNA-656

54) microRNA-1247.

For the avoidance of doubt, each of the 54 microRNAs listed above are 14q32 microRNAs—that is, they are encoded by sequences located within the 14q32 locus of the human chromosome. Additionally, each of microRNAs 1-54 above may be termed a “vasoactive microRNA”.

Accordingly, the invention provides modulators, for example inhibitors, of one or more of the 14q32 microRNAs listed as 1-54 above, for modulating vascular re-modelling processes and/or for use in treating or preventing vascular disorders.

Furthermore, the invention provides a method of modulating vascular re-modelling processes and/or of treating or preventing a vascular disorder, said method comprising administering modulators, for example inhibitors, of one or more of the 14q32 microRNAs listed as 1-54 above to a subject in need thereof. The modulators may be administered in a therapeutically effective amount. The subject in need thereof may be a human or animal subject.

The present invention provides modulators (for example inhibitors) of one or more of miR-329, miR-494, miR-487b and/or miR-495 for use in treating vascular disorder/disease and/or modulating vascular re-modelling processes. More specifically modulators, for example inhibitors, of these microRNAs may be for use in methods of inhibiting atherosclerosis (specifically modulators of mir-329, miR-494 and/or miR-495), reducing blood cholesterol levels (specifically modulators of miR-494 and/or miR-495), increasing plaque stability (specifically modulators of miR-329, miR-494 and/or miR-495) and inducing angiogenesis and/or arteriogenesis (specifically modulators of miR-329, miR-494, miR-487b and/or miR-495). Again it should be noted that uses and methods of this invention which exploit modulators of miR-329, miR-494 and/or miR-495 may not only bring about the induction of arteriogenesis and/or angiogenesis but also simultaneously inhibit atherosclerosis and/or restenosis.

Modulators of miR-487b may not be used to modulate hypertension induced remodelling of the aorta.

It should be understood that microRNA clusters equivalent to the cluster located at 14q32 of human chromosome 14, are located on chromosome 12 in mice and 6 in Rats. Accordingly, insofar as the invention relates to methods and uses which exploit vasoactive microRNAs of the human genome, it should be understood that the invention extends to methods and uses of the equivalent microRNAs from other mammalian chromosomal loci.

While investigating the arteriogenesis phenomenon, the inventors noted the expression patterns of certain microRNAs differed. For example it was noted that some microRNAs showed a rapid decrease in expression following the onset of arteriogenesis—these were termed the “fast responders”. Others showed a moderate initial increase in expression followed by strong decrease at around 3-7 days following the onset of arteriogeneis. After about 7 days, the expression level of these microRNAs again rose. These microRNAs were termed “slow responders”. Others exhibited only a slight decrease in expression throughout the whole period of arteriogenesis—these are the “non-responders”. Table 1 below identifies the fast, slow and non-responder microRNAs.

TABLE 1
14q32 MicroRNAs

| Fast | Slow | Non |
|---|---|---|
| Responder | Responder | Responder |
| 337 | 770 | 493 |
| 432 | 673 | 540 |
| 494 | 665 | 431 |
| 666 | 433 | 136 |
| 487b | 127 | 370 |
| 134 | 434 | 379 |
| 453 | 341 | 380 |
| 154 | 882 | 758 |
| 409 | 411 | 543 |
| 410 | 299 | 495 |
| | 323 | 376c |
| | 329 | 539 |
| | 679 | 544 |
| | 667 | 485 |
| | 654 | 496 |
| | 376b | 541 |
| | 376a | |
| | 300 | |
| | 381 | |
| | 382 | |
| | 668 | |
| | 377 | |
| | 412 | |
| | 369 | |

Based on the above, and when treating certain diseases or modulating vascular re-modelling processes such as arteriogenesis, one of skill might adopt a phased approach to the modulation of the 14q32 microRNAs. For example and with reference to uses and methods which aim to increase or promote arteriogenesis, one might opt to first modulate the expression of one or more fast responder type microRNAs (as identified in Table 1) and then one or more of the slow responder microRNAs. Optionally, one might also continually inhibit one or more of the non-responder microRNAs.

Modulators suitable for use in this invention include inhibitors of the 14q32 microRNAs, including the specific 14q32 microRNAs described herein.

Inhibitors suitable for use in this invention may include, for example small organic/inorganic molecules, proteins, peptides, amino acids, nucleic acids (comprising RNA, DNA and/or synthetic or peptide based nucleic acids, including PNA), carbohydrates, lipids, antibodies (including antigen binding fragments thereof) and the like.

In particular, the term “inhibitor” applies to oligonucleotides including, DNA and/or RNA based antisense oligonucleotides, which comprise molecules/sequences which bind or are complementary to a particular target microRNA. Oligonucleotide based inhibitors may be referred to as “gene silencing oligonucleotides” (GSOs). Thus the invention may exploit GSO type inhibitor compounds.

For example, the term “inhibitor” may encompass synthetic oligonucleotide-based compounds described in WO2012/135152 (the disclosure of which is incorporated herein in its entirety) and by Bhagat et al., (J. Med. Chem., 2011, 54, 3027-3036).

Antisense oligonucleotides for use in this invention may comprise (nucleic acid, DNA and/or RNA or synthetic/modified bases as described below) sequences which are complementary to all or part of one or more of the 14q32 microRNA sequences (or equivalent microRNA sequences in other genomes, including the murine and rat genomes) disclosed herein. For example, antisense oligonucleotides for use in this invention may comprise sequences which are complementary to a seed sequence of the target microRNA. By comprising sequences complementary to sequences of the target microRNA(s), antisense oligonucleotide inhibitors can form duplexes with the target microRNA—the formation of such duplexes prevents the microRNA from binding its intended (mRNA) target.

Oligonucleotide and/or antisense oligonucleotides of this invention may include, for example antagomir and/or blockmir type inhibitors as well as inhibitory RNA molecules.

One of skill will appreciate that an “antagomir” is a single-strand chemically-modified ribonucleotide having at least a partially complementary sequence to a target microRNA, such as, for example a target vasoactive microRNA sequence of this invention.

An oligonucleotide or antisense oligonucleotide for use in this invention may comprise one or more modified oligonucleoticles and/or one or more chemical modifications. For example, an antisense oligonucleotide microRNA inhibitor for use in this invention may comprise peptide nucleic acid (PNA). The antisense oligonucleotide may include other chemical modifications, for example, sugar modifications such as 2′-O-alkyl (e.g., 2′-O-ethyl and 2′-O-methoxyethyl), 2′-fluoro and 4′-thio modifications as well as backbone modifications such as phosphorothiate, morpholinos or phosphonocarboxy linkages (e.g., U.S. Pat. No. 6,693,187 and U.S. Pat. No. 7,067,641, the contents of which are incorporated herein by reference). The oligonucleotides of this invention may comprise, or further comprise, modifications aimed at improving the stability of the molecule and/or its in vivo delivery. For example, an oligonucleotide/antisense oligonucleotide may comprise a cholesterol moiety. The oligonucleotides of this invention may comprise a 2′-O-methoxyethyl “gapmer” comprising 2′-O-methoxyethyl-modified ribonucleotides at the 5′-end and 3′-end and at least 10 deoxyribonucleotides therebetween. The “gapmer” can trigger RNase I-dependent degradation mechanism of an RNA target.

An oligonucleotide or antisense oligonucleotide of this invention may comprise one or more locked nucleic acid(s) (LNA). LNA is a modified ribonucleotide with a “locked form” in which the ribose moiety is modified with an extra bridge connecting the 2′ oxygen and 4′ carbon. LNA nucleotides can be mixed with DNA or RNA residues to form an antisense oligonucleotide for use in this invention.

An inhibitor molecule (for example anti-sense oligonucleotide) of this invention may comprise at least about 5 to about 50 nucleotides, for example at least about 10 to about 40 nucleotides or at least about 15 to about 30 nucleotides. Suitable antisense inhibitor molecules may comprise at least about 20 to about 25 nucleotides, for example about 22 nucleotides. One of skill will appreciate that an antisense oligonucleotide molecule which is to find application as a microRNA inhibitor, may comprise a number of nucleotides corresponding to the number of nucleotides present in the target microRNA sequence.

Inhibitors of this invention may comprise inhibitory RNA molecules, which inhibitory RNA molecules comprise sequences which are complementary to the target microRNA sequence. Suitable inhibitory RNA inhibitors may small interfering RNA (siRNA), short hairpin RNA (shRNA) and ribonucleic acid enzyme (ribozyme).

In addition to exploiting 14q32 microRNA inhibitors, one of skill will appreciate that this invention may exploit or further exploit compounds or molecules which modulate some aspect of the expression, function and/or activity of genes which are the target of the 14q32 (vasoactive) microRNAs. It should be understood that since the role of microRNA is to control gene expression, the modulation of microRNA expression leads, in turn, to modulation of gene expression. As such, modulators, including compounds which increase the expression, function and/or activity of one or more of the 14q32 gene targets, may be used to achieve the modulation of vascular re-modelling processes and/or the treatment and/or prevention of vascular disorders, diseases, conditions and/or syndromes.

Suitable modulators may comprise molecules which enhance the expression, function and/or activity of a gene targeted by a 14q32 microRNA. Modulators of this type may affect (increase) the activity of a promoter associated with a particular gene. Modulators which enhance gene expression may comprise the protein product of the gene in question (or a functional fragment thereof) or a nucleic acid sequence encoding the same. A nucleic acid sequence for use as a modulator may be provided in the form of an expression vector.

Modulators for use in this invention may comprise molecules which inhibit or suppress the expression, function and/or activity of a gene targeted by a 14q32 microRNA. Modulators of this type may inhibit (decrease) the activity of a promoter associated with a particular gene. Inhibitory modulators may further comprise oligonucleotides, for example antisense oligonucleotides or synthetically prepared microRNA molecules designed to suppress, ablate or inhibit the expression of a particular gene. Techniques and “apps” for designing inhibitor (nucleic acid based) molecules of this type are well known to those skilled in the field and further information may be obtained from Rebecca Schwab et al (2006: Highly Specific Gene Silencing by Artificial MicroRNAs in Arabidopsis Plant Cell 18: 1121-1133 and Stephan Ossowski et al (2008: Gene silencing in plants using artificial microRNAs and other small RNAs The Plant Journal 53 (4), 674-690

Modulators for use in this invention may take the form of antibodies which exhibit an affinity or specificity for the protein product (or an epitope thereof) of a gene the expression of which may be (at least partly) controlled by a 14q32 microRNA. Antibodies may be polyclonal and/or monoclonal and the techniques used to generate antibodies are well known in the art and may involve the use of animal immunisation protocols (for the generation of polyclonal antibodies) or the generation of hybridomas (for generating monoclonal antibodies). Further information on the preparation and use of polyclonal and/or monoclonal antibodies may be obtained from Using Antibodies: A Laboratory Manual by Harlow & Lane (CSHLP: 1999) and Antibodies: A Laboratory Manual by Harlow & Lane (CSHLP: 1988)—both of which are incorporated herein by reference.

Thus in a further aspect, the invention provides modulators of one or more genes which are targeted or at least partly regulated by one or more 14q32 microRNAs for modulating vascular re-modelling processes and/or for use in treating or preventing vascular disorders.

The modulators of this invention may be provided as compositions comprising one or more excipients, carriers and/or diluents. The compositions may take the form of pharmaceutical compositions and thus may be sterile and/or comprise pharmaceutically acceptable excipients, carriers and/or diluents.

### DETAILED DESCRIPTION

The present invention will now be described in detail with reference to the following Figures which show:

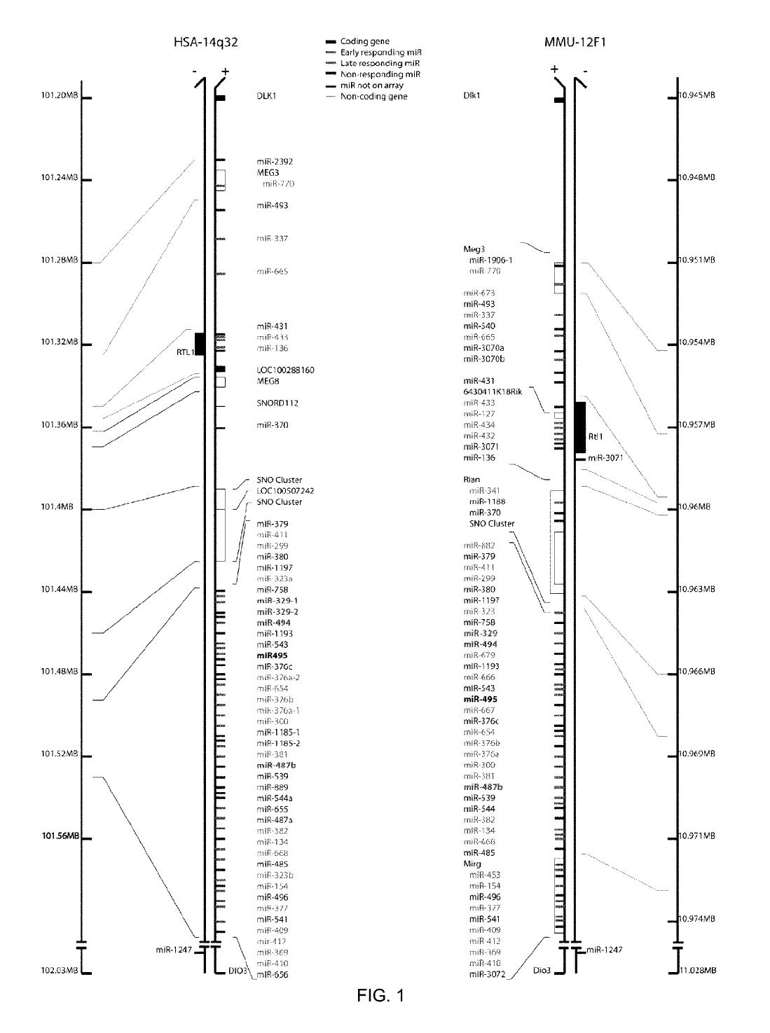

FIG. 1. A schematic overview of the human 14q32 and murine 12F1 loci. Colours indicate whether the murine miRs were early, late or non-responders after hind limb ischemia induced by single ligation of the femoral artery in healthy C57Bl/6 mice.

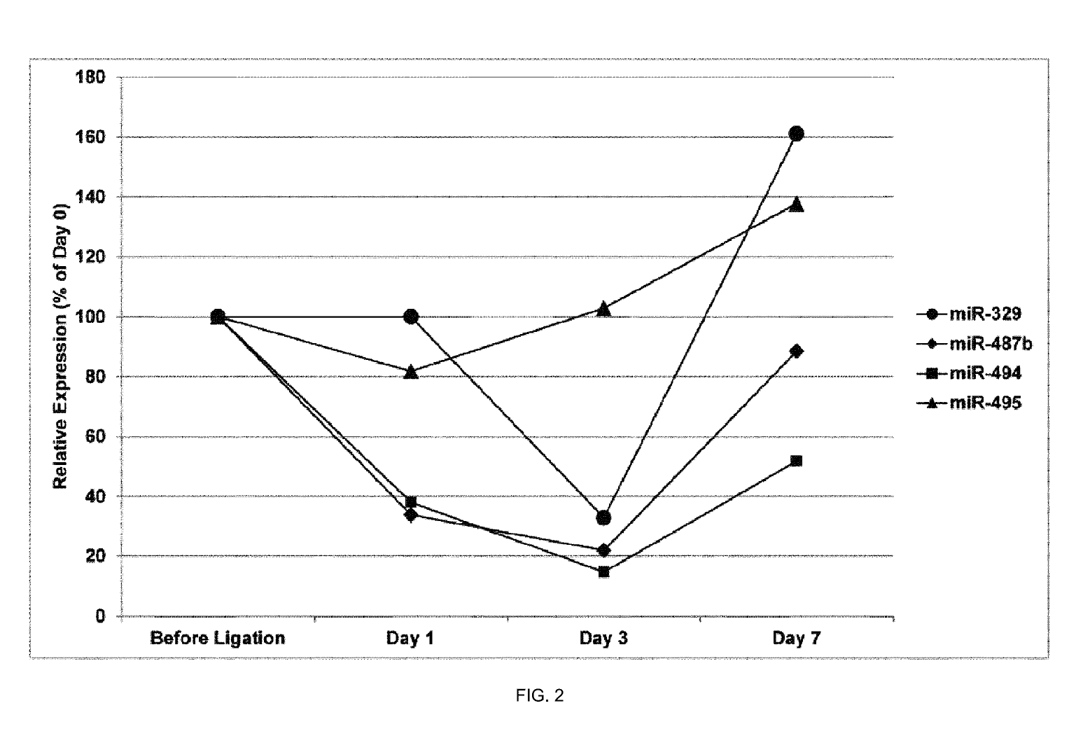

FIG. 2. 14q32 miR expression during effective arteriogenesis. MicroArray analyses of total RNA isolated from left adductor muscles of mice (4 mice per time point) before and at 1, 3 and 7 days after single ligation of the left femoral artery. Expression levels of miR-329, miR-487b, miR-495 and miR-494 are expressed as percentages of their individual expression levels before femoral artery ligation.

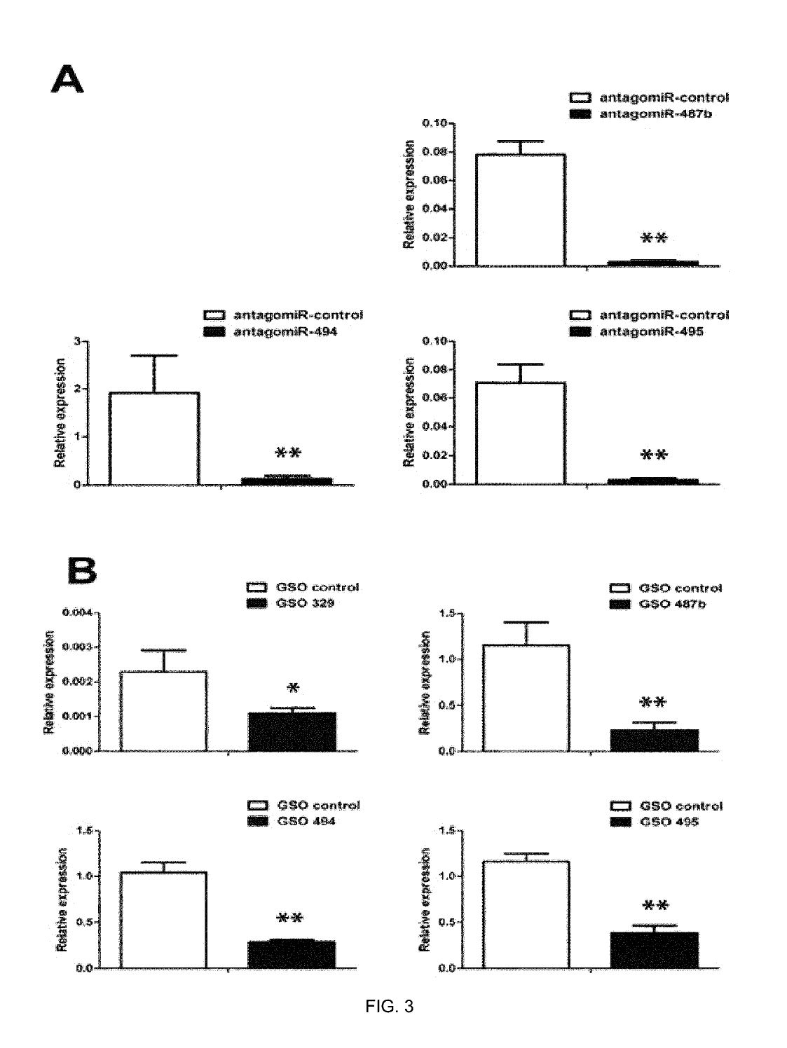

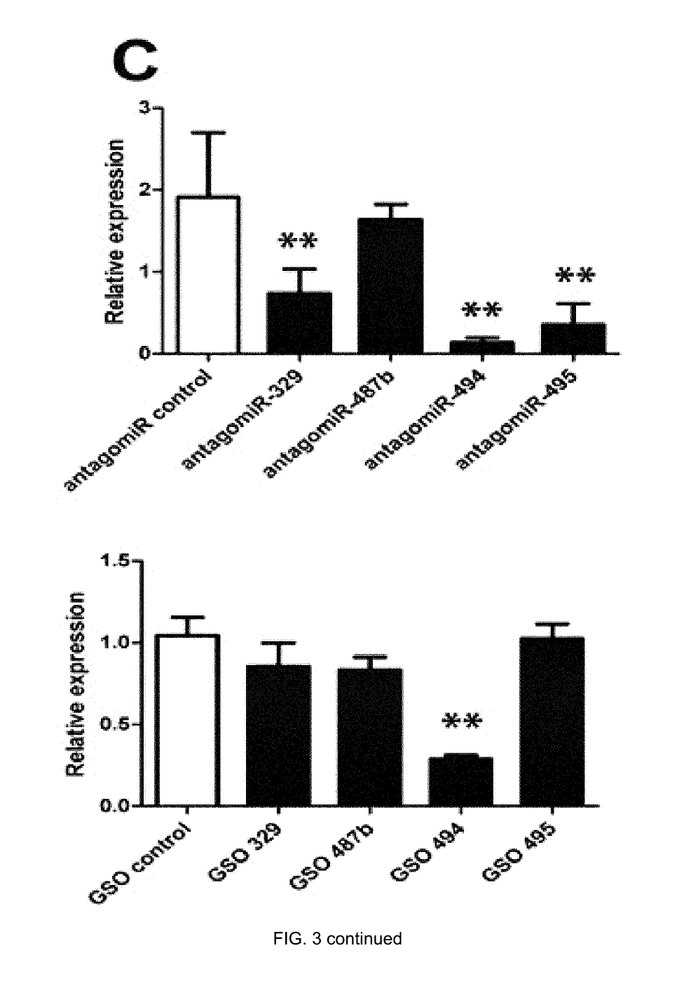

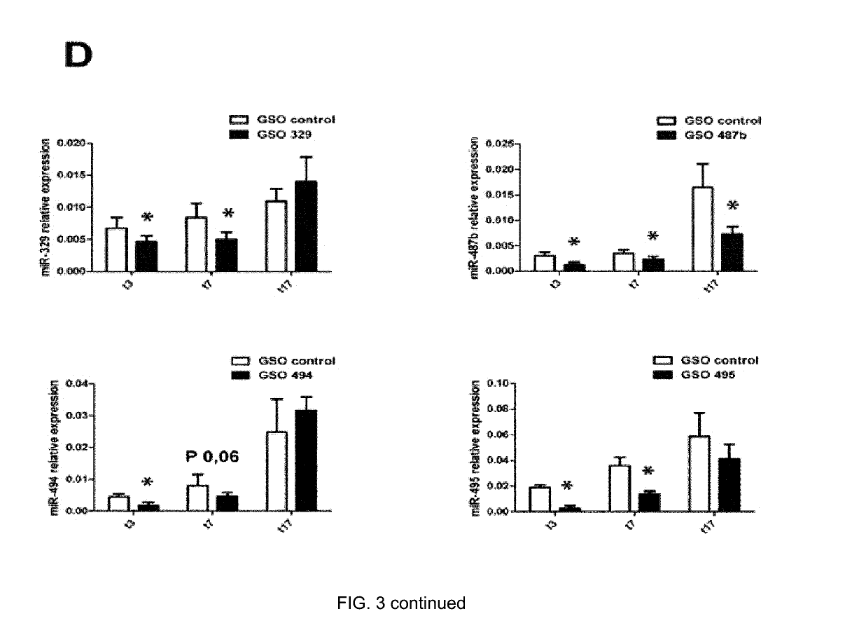

FIG. 3. MiR inhibition in primary human arterial fibroblasts and in adductor muscle tissue of mice subjected to Hind Limb Ischemia. (A) Inhibition of individual 14q32 miRs in primary HUAFs by antagomiRs (5 ng/μl). (B) Inhibition of individual 14q32 miRs in primary HUAFs by GSOs (5 ng/μl). (C) Expression levels of individual 14q32 miRs in primary HUAFs after treatment with antagomiR-494 (5 ng/μl)(Upper Panel) and expression levels of individual 14q32 miRs in primary HUAFs after treatment with GSO-494 (5 ng/μl) (Lower Panel). Mean expression levels, from at least 3 independent experiments, relative to miR-191 are shown here ±SEM. *P<0.05, ** P<0.01. (D) Expression of 14q32 miRs in adductor muscle tissue of C57Bl/6 mice, subjected to double ligation of the left femoral artery at days 4, 8 and 18 after treatment with GSO-329, GSO-487b, GSO-494 and GSO 495 compared to GSO control. Per group, adductor muscle tissue of 3 mice was used. Mean expression levels relative to let-7C are shown here ±SEM. *P<0.05.

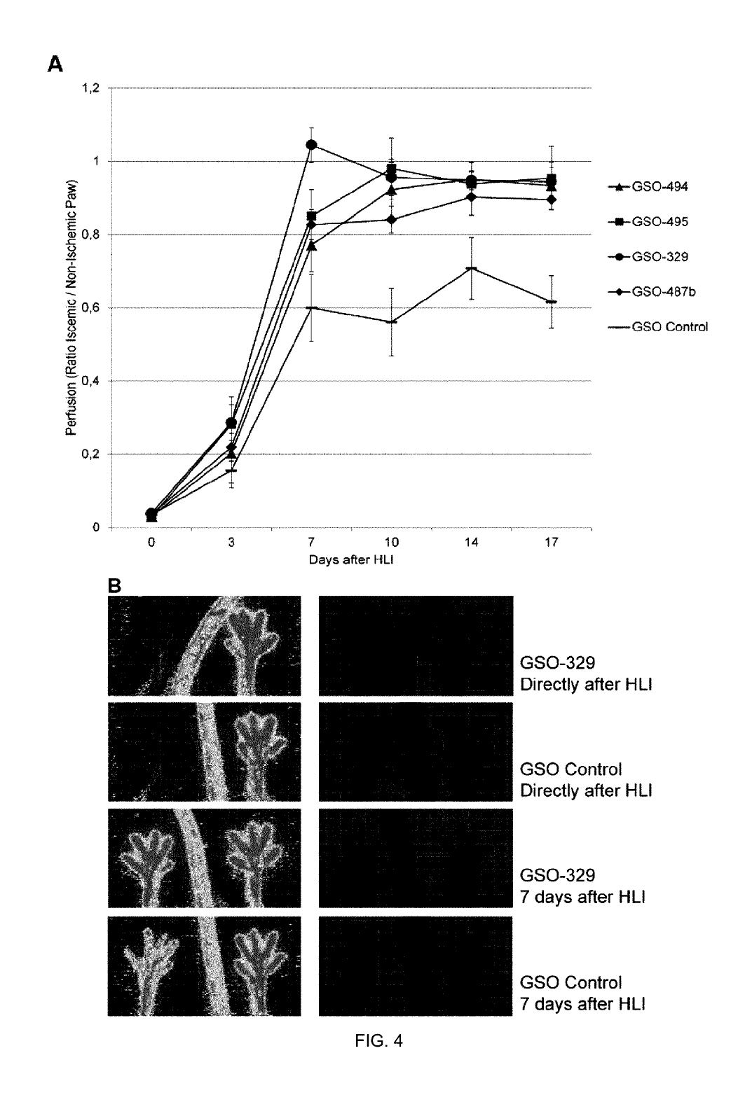

FIG. 4. Blood flow recovery after in vivo 14q32 miR inhibition. (A) Quantification of LDPI measurements over time in mice (11 per group) treated with either PBS or GSOs (1 mg/mouse). Data are calculated as the ratio of the ischemic over the non-ischemic paw. Data are presented as mean±SEM. (B) Representative LDPI images of paws directly and 7 days after induction of HLI in the left limb of mice treated with either GSO Control or GSO-329.

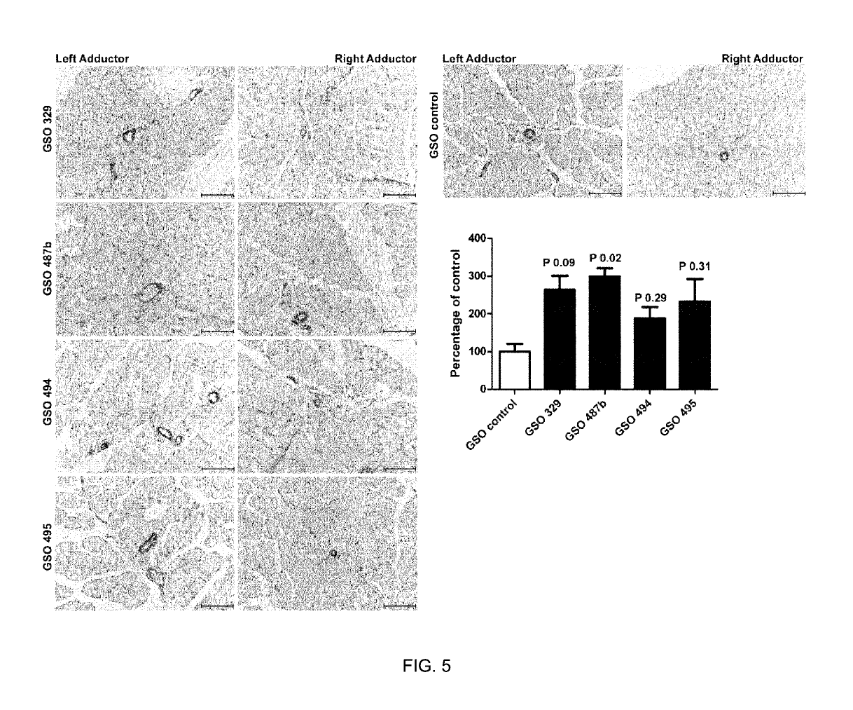

FIG. 5. In vivo arteriogenesis after 14q32 miR inhibition. Representative images of α-SMA staining in right (untreated) and left (ligated femoral artery) adductor muscle tissues of mice treated with GSOs and quantification of the increase in diameter of α-SMA⁺ arterioles, relative to the increase in mice treated with GSO Control. Per group, left and right adductor muscles of 11 mice were included. From each muscle, 8 sections were used and from each section, 1 representative photograph was used. The scale bar represents 100 μm. Data are presented as mean±SEM.

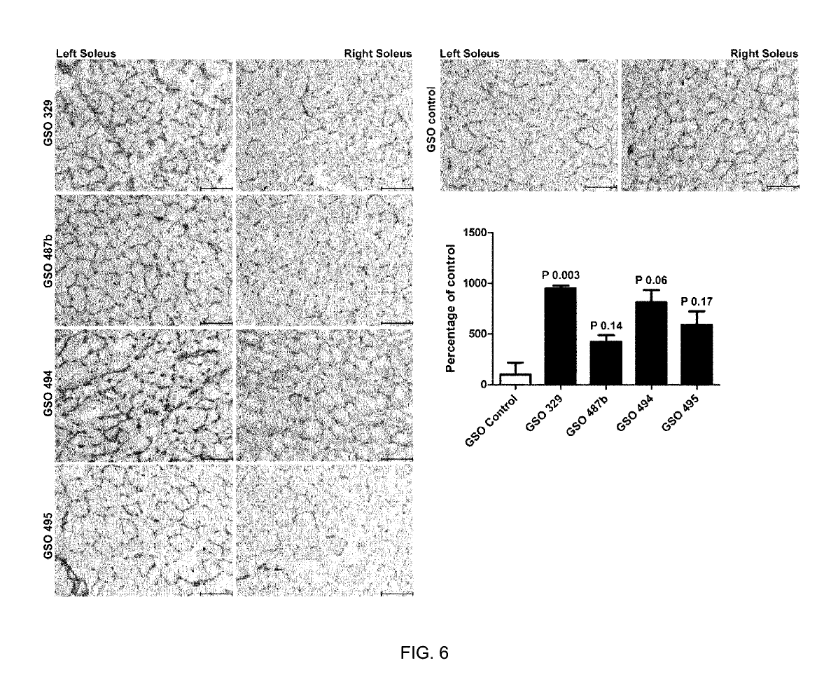

FIG. 6. In vivo angiogenesis after 14q32 miR inhibition. Representative images of CD31 staining in right (normoxic) and left (ischemic) soleus muscles of mice treated with GSOs. and quantification of the increase in CD31⁺ area between right and left soleus muscles, relative to the increase in mice treated with GSO Control. Per group, left and right soleus muscles from 3 mice were included. From each muscle, 6 sections were used and from each section, 1 representative photograph was used. The scale bar represents 100 μm. Data are presented as mean±SEM.

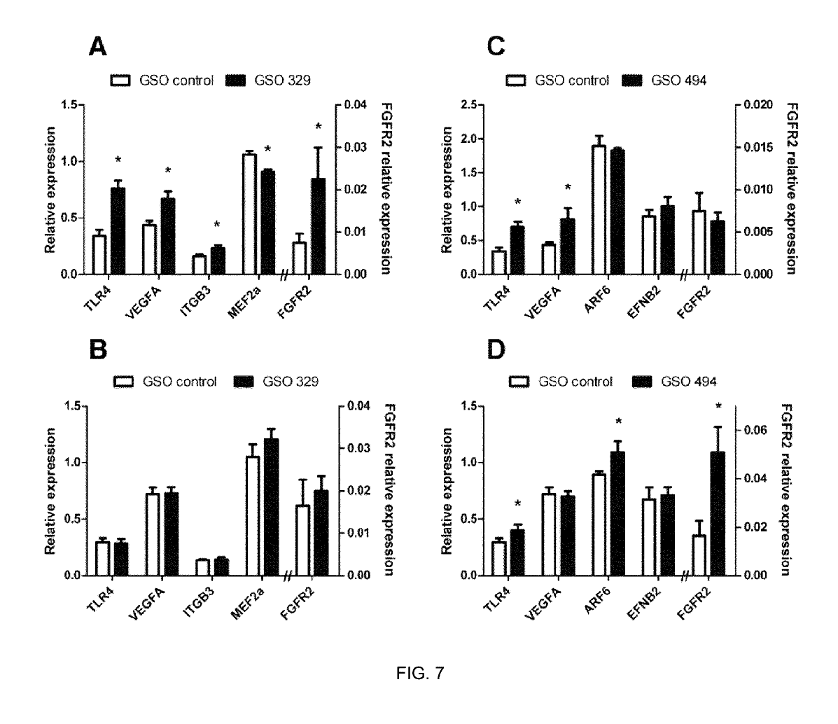

FIG. 7. In vivo regulation of putative target genes. Expression levels of putative target genes for miR-329 relative to HPRT1 in adductor muscle tissue of mice treated with GSO-329 at days 3 (A) and 7 (B) after HLI. Expression levels of putative target genes for miR-494 relative to HPRT1 in adductor muscle tissue of mice treated with GSO-494 at days 3 (C) and 7 (D) after HLI. Per group, adductor muscle tissue of 3 mice was used. Data are presented as mean±SEM. *P<0.05.

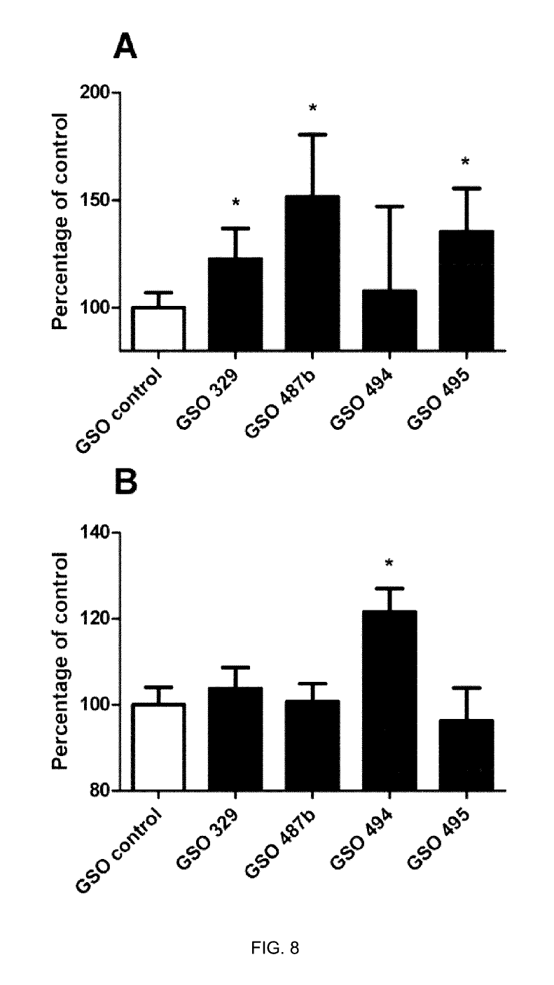

FIG. 8. In vitro effects of 14q32 miR inhibition. (A) Proliferation of HUAECs after GSO treatment (15 ng/μl) measured by ³H-thymidine incorporation relative to GSO Control. (B) Proliferation of HUAFs after GSO treatment (10 ng/μl) measured by ³H-thymidine incorporation relative to GSO Control. Data are presented as mean±SEM and represent at least 3 independent experiments. *P<0.05.

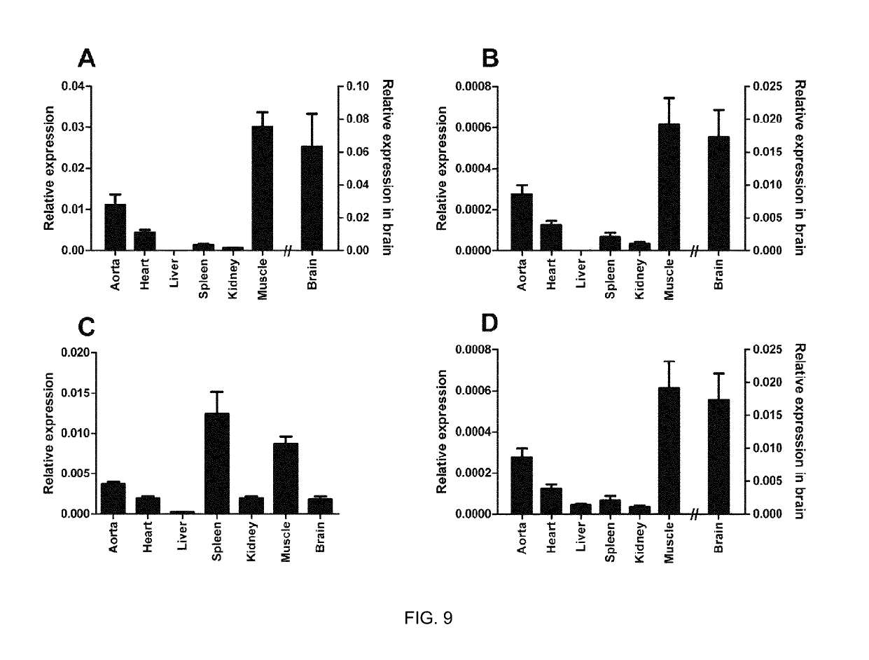

FIG. 9: Expression of 14q32/12F1 miRs. Expression levels of miR-329, miR-487b, miR-494 and miR-495, relative to Let-7c in aorta, heart, spleen, kidney, skeletal muscle and brain and relative to Let-7c and miR-122 in the liver in healthy, adult C57Bl/6 mice. Tissues/organs from 9 mice were included and pooled per 3 animals. Data are presented as mean±SEM.

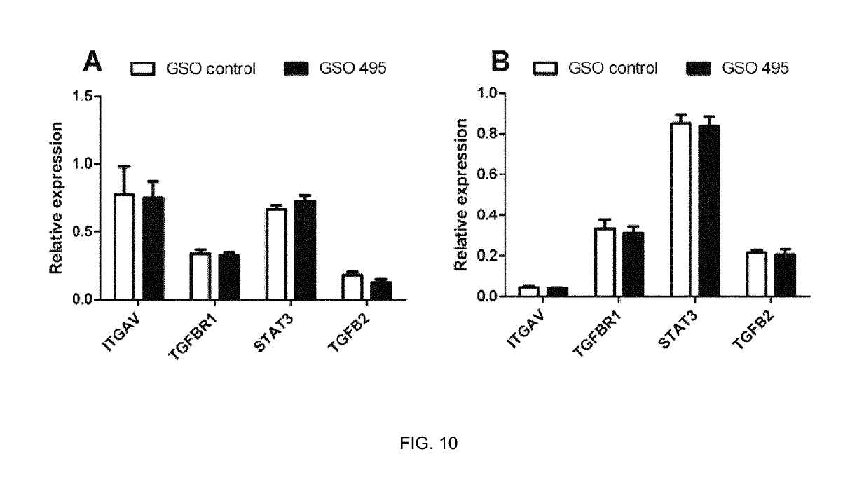

FIG. 10: In vivo regulation of putative target genes of miR-495. Expression levels of putative target genes for miR-495 relative to HPRT1 in adductor muscle tissue of mice treated with GSO-495 at days 3 (A) and 7 (B) after HLI. Per group, adductor muscle tissue of 3 mice was used. Data are presented as mean±SEM.

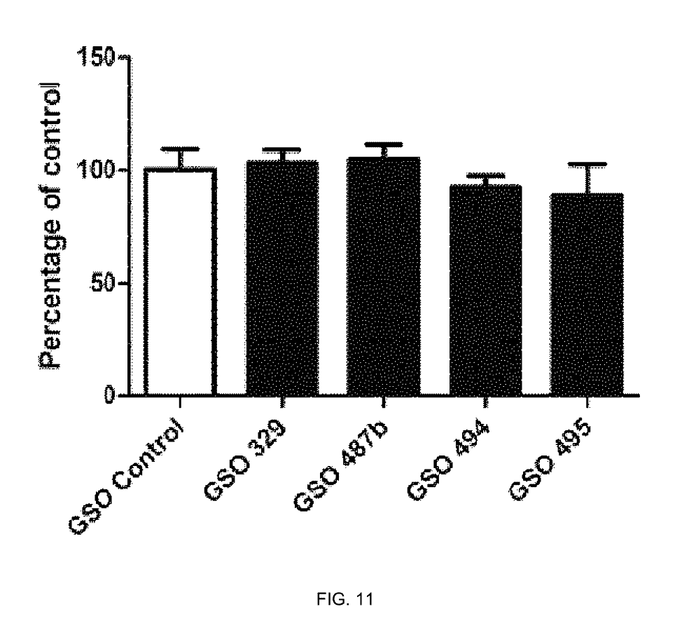

FIG. 11: In vitro effects of 14q32 miR inhibition. Proliferation of HUASMCs after GSO treatment (10 ng/μl) measured by ³H-thymidine incorporation, relative to GSO Control. Data are presented as mean±SEM and represent at least 3 independent experiments.

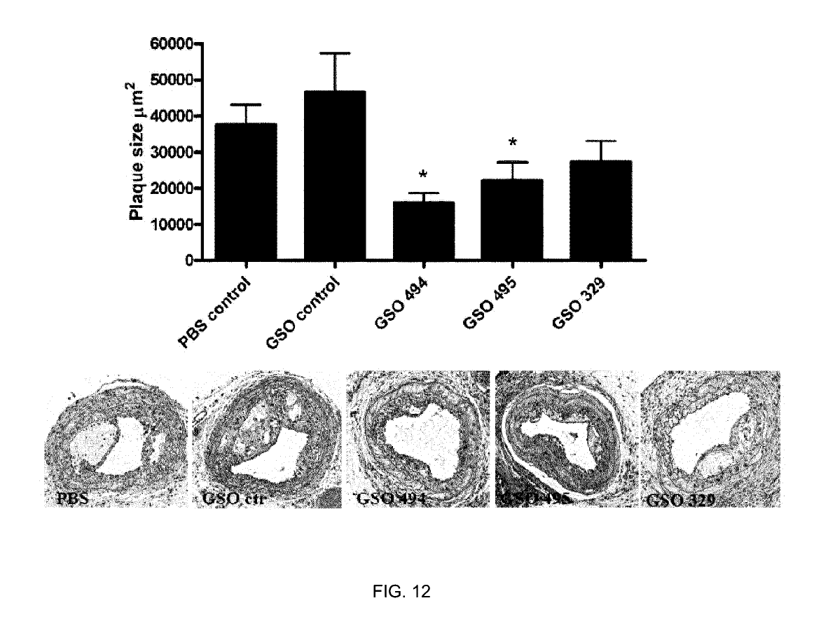

FIG. 12. Inhibition of miR-494, -495 and -329 reduces atherosclerotic lesion formation. Inhibition of miR-494 and -495 by GSOs leads to a reduction of atherosclerotic plaque formation in carotid arteries of ApoE^{-/-} mice. GSO-329 shows a trend towards a decrease in lesion size. No significant differences were found between PBS and GSO control. The micrographs show representative images of each treatment group (10×).

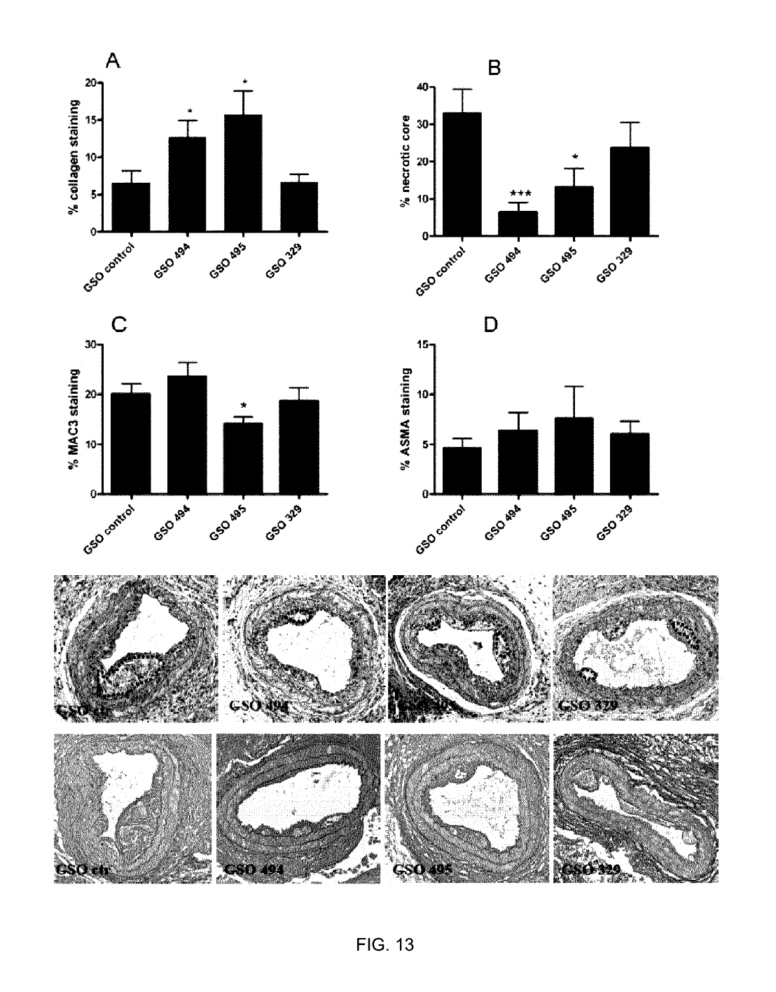

FIG. 13. Treatment of GSO-494, -495 leads to a profound increased stable phenotype of atherosclerotic lesions. Effect of inhibition of miR-494, -495 and -329 on plaque morphology and lesion stability. A. Collagen content in the lesions was increased in the groups treated with GSO 494 and 495 (*P<0.05). B. Necrotic core size was defined as an a-cellular area rich of debris and was measured as a percentage of total plaque area. Interestingly, inhibition of miR-494 and -495 led to a decrease of necrotic core size (*P<0.05, ***P<0.001), which is, together with the increased collagen content, suggestive of an increased stable phenotype. C. Macrophages were visualized by MAC-3 antibody and expressed as a percentage of stained area in the intima. Only mice treated with GSO-495 showed a decrease in lesional macrophages (*P<0.05). D. No differences were found in the smooth muscle cell content of the atherosclerotic plaques. The micrographs show representative images of necrotic core size in all treated groups (10×).

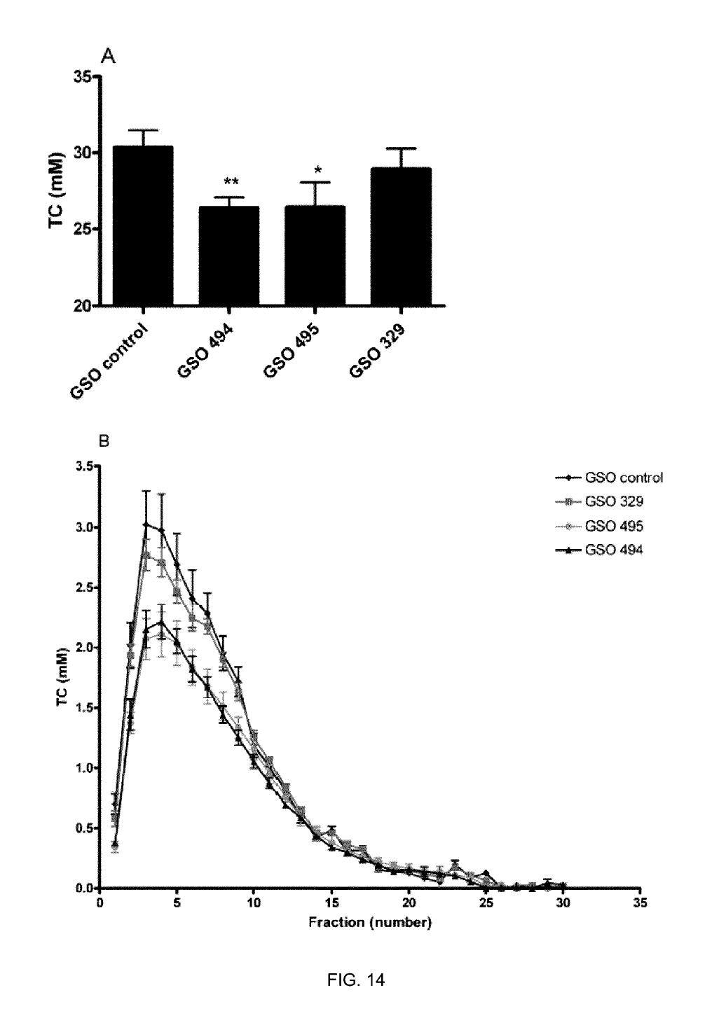

FIG. 14. Reduction in cholesterol levels after inhibition of miR-494 and -495. Cholesterol levels were reduced after treatment with GSO-494 and GSO-495 at time of sacrifice, after 6 weeks of western type diet (A). AKTA-FPLC analysis revealed a clear decrease in VLDL/LDL level (B). * P<0.05; **P<0.01.

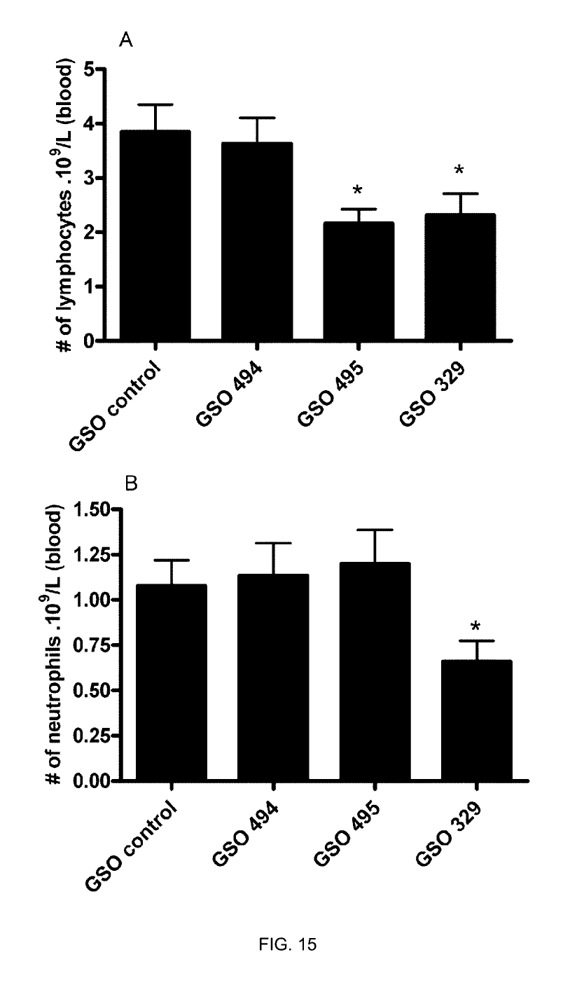

FIG. 15. Changes in blood lymphocytes and neutrophils after GSO treatment. Inhibition of miR-329 and miR-495 leads to a decrease in absolute amount of lymphocytes in the blood at day 28 (A). Furthermore, inhibiting miR-329 also resulted in a decrease of neutrophils (B). *P<0.05.

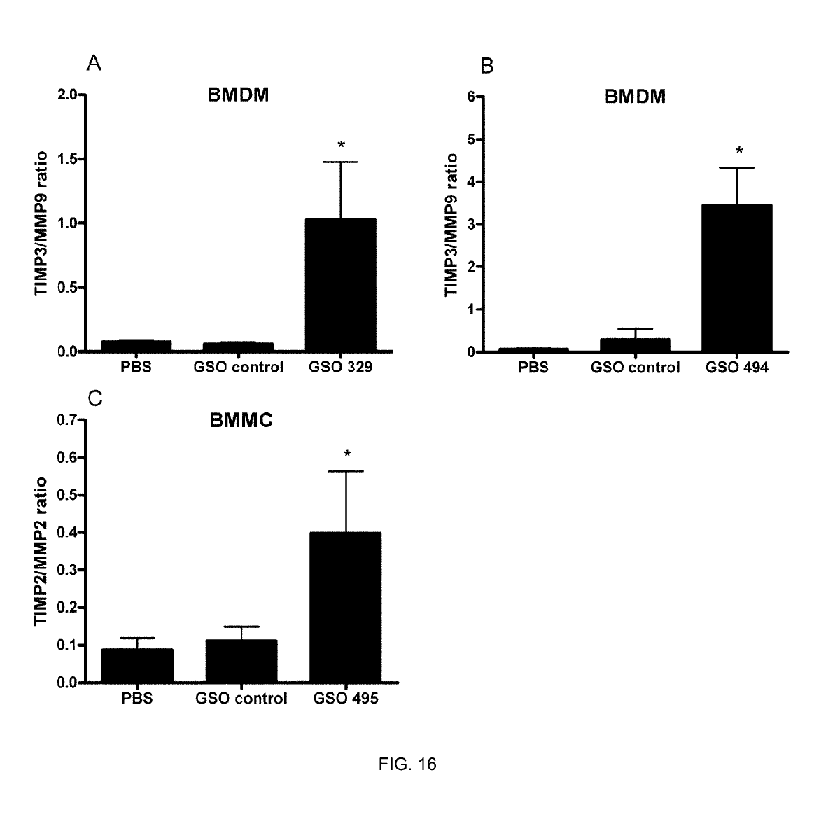

FIG. 16. Increased TIMP/MMP ratio after GSO treatment. After treatment with GSOs the expression level of TIMP3, a target gene of miR-329 and miR-494, was significantly increased in BM derived macrophages. TIMP2, a target gene of miR-495, was increased in BM derived mast cells after treatment with GSO-495. MMP levels remained unchanged after GSO treatment, resulting in a positive TIMP/MMP ratio. *P<0.05.

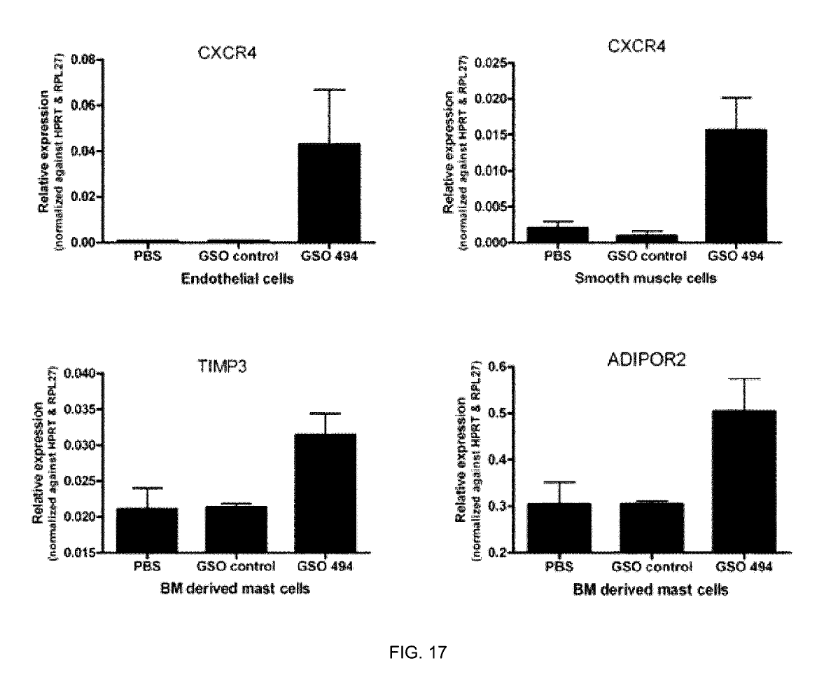

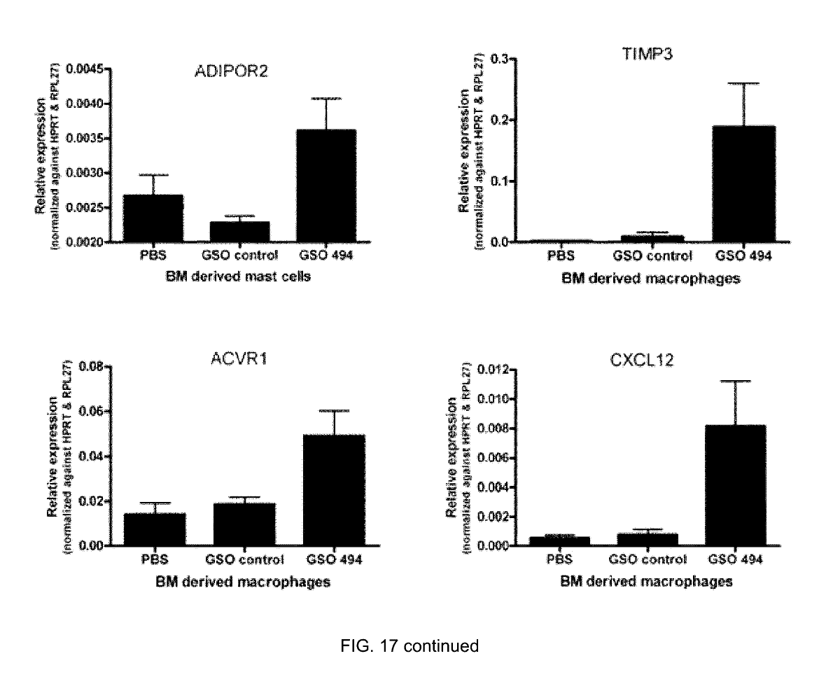

FIG. 17. In vitro regulation of target gene expression after GSO-induced inhibition of miR-494 in murine endothelial cells, smooth muscle cells, bone marrow (BM) derived mast cells and BM derived macrophages.

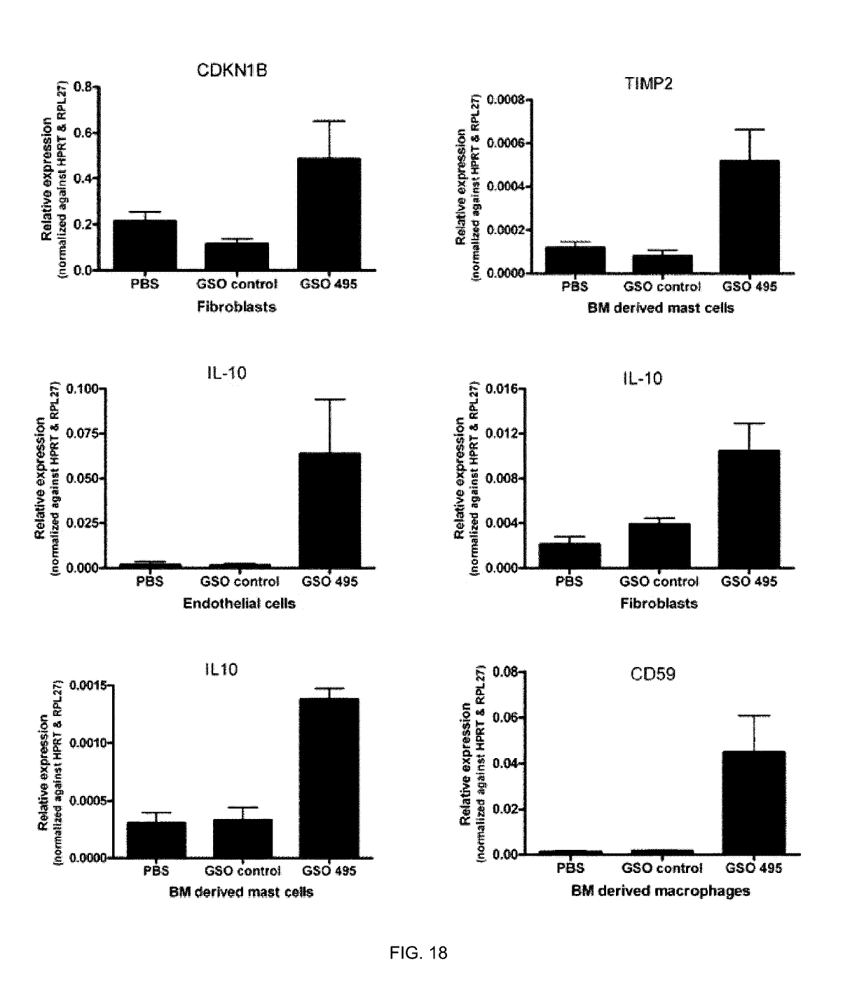

FIG. 18. In vitro regulation of target gene expression after GSO-induced inhibition of miR-495 in murine endothelial cells, fibroblasts, bone marrow (BM) derived mast cells and BM derived macrophages.

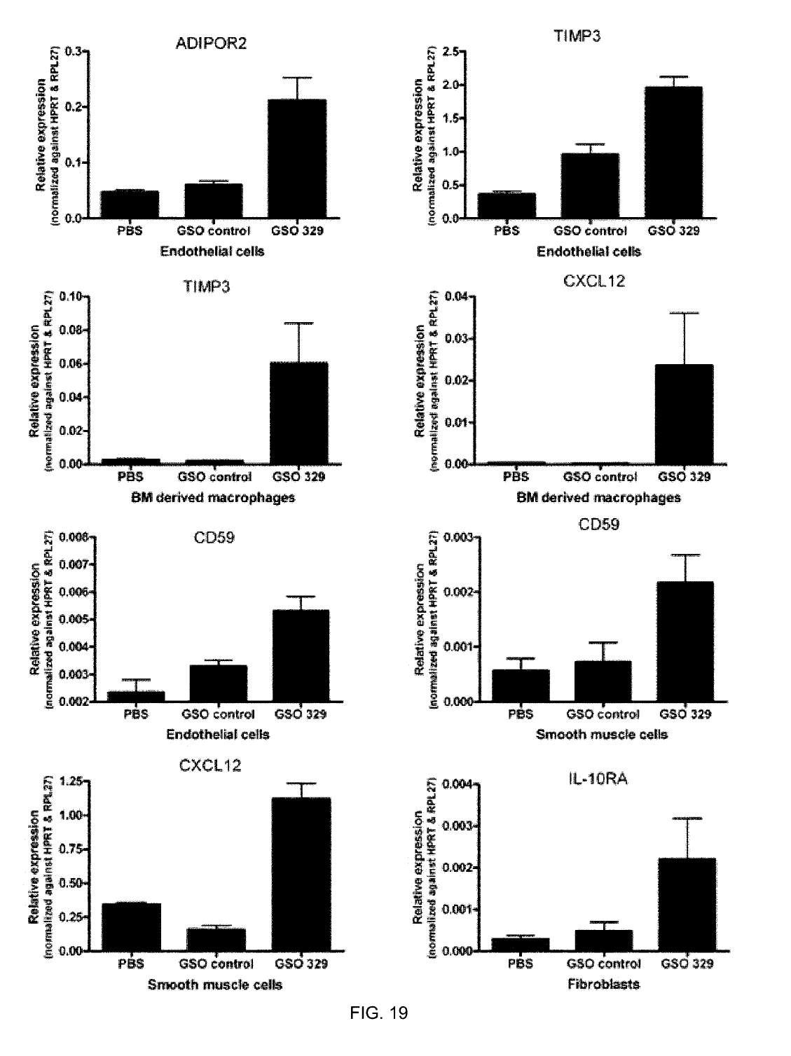

FIG. 19. In vitro regulation of target gene expression after GSO-induced inhibition of miR-329 in murine endothelial cells, smooth muscle cells, fibroblasts and bone marrow (BM) derived macrophages.

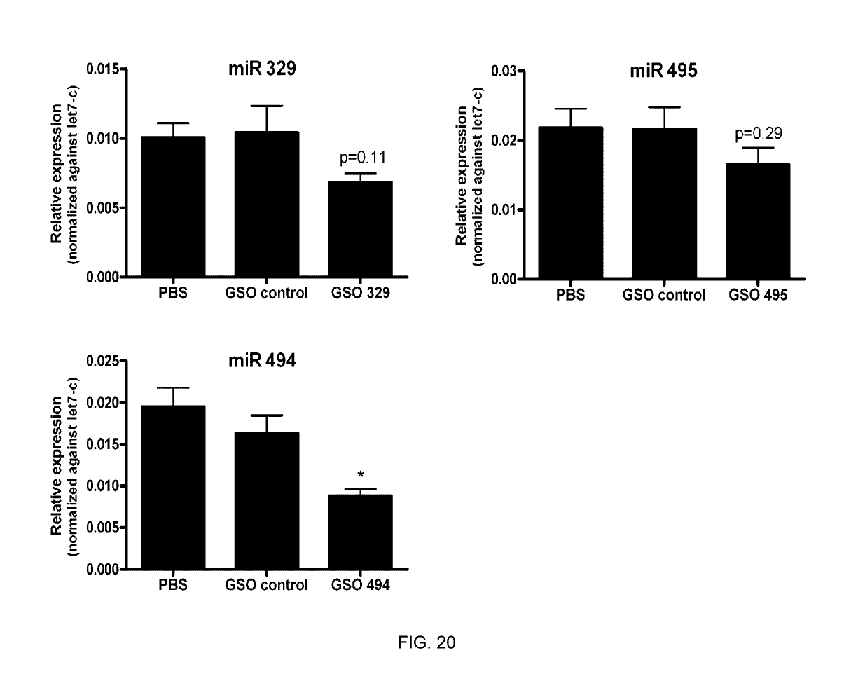

FIG. 20. Expression of miR-329, miR-495 and miR-494 in the carotid arteries of mice at three days after injection of 1 mg GSO, GSO-control or PBS.

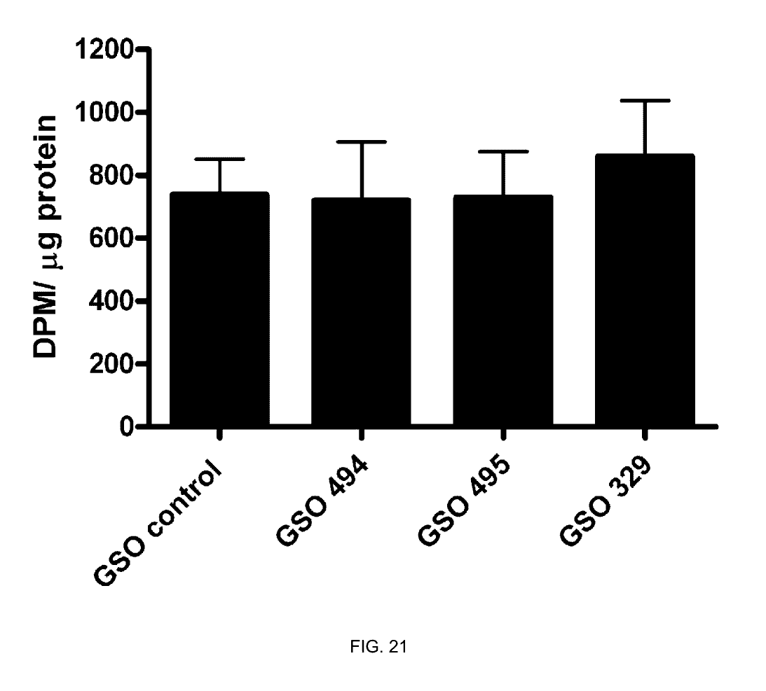

FIG. 21. Collagen synthesis in vitro was measured by ³H-proline incorporation in murine smooth muscle cells after GSO-induced inhibition of miR-494, miR-495 or miR-329, compared to GSO-control. Results are expressed as disintegrations per minute (DPM), relative to total protein synthesis.

### EXAMPLE 1

### Materials & Methods

### Reverse Target Prediction

To identify miRs that are involved in arterio- and angiogenesis, an in silico reverse target prediction (RTP) was performed. A selection was made of 127 genes known from literature and previous studies within our group to play important roles in arterio- and angiogenesis (Table 51). To ensure the master switch character of the identified miRs, we selected target genes covering all aspects of vascular remodeling; endothelial activation, smooth muscle cell proliferation, extracellular matrix rearrangement, chemo- and cytokines and their receptors, growth factors and their receptors, the natural killer complex, pro-arteriogenic and pro-angiogenic transcription factors and signaling molecules (Table 51). We then used www.targetscan.org to, for each individual gene, generate a list of all miRs predicted to target these 127 genes. Each list, no restrictions were applied, was copied into a spreadsheet and for each miR we simply counted the number of times it was present in the file.

TABLE S1
Reverse Target Prediction 1; Target genes selected from literature.

| | | PubMed Hits (N) | PubMed Hits (N) |
|---|---|---|---|
| | | on Search | on Search |
| | | Term “gene | Term “gene |
| | | name/protein | name/protein |
| | Target | name/alias + | name/alias + |
| Process | Gene | arteriogenesis” | angiogenesis” |
| Endothelial | NOS3 | 30 | 649 |
| Activation | NOS2 | 8 | 288 |
| | EDN1 | 2 | 373 |
| | VEGFA | 16 | 3130 |
| | BMP4 | 0 | 46 |
| | KLF2 | 1 | 20 |
| Smooth | PTK2 | 2 | 282 |
| Muscle Cell | ITGA5 | 2 | 7 |
| Proliferation | ITGAV | 1 | 13 |
| | ITGB1 | 1 | 6 |
| | ITGB2 | 1 | 6 |
| | ITGB3 | 3 | 2 |
| | ITGAM | 2 | 7 |
| | ITGAL | 0 | 3 |
| | GJA4 | 3 | 24 |
| | GJA5 | 5 | 15 |
| | GJA1 | 5 | 37 |
| | GJC1 | 0 | 2 |
| Adhesion | ICAM1 | 7 | 274 |
| Molecules | VECAM1 | 10 | 9 |
| | PECAM1 | 20 | 1193 |
| | CCL2 | 45 | 439 |
| | CDH5 | 5 | 366 |
| | SELE | 2 | 190 |
| | SELP | 2 | 96 |
| | CCR2 | 6 | 54 |
| Extracellular | MMP2 | 3 | 207 |
| Matrix | MMP9 | 1 | 183 |
| | SERPINE1 | 3 | 399 |
| | PLAT | 0 | 48 |
| | PLAU | 3 | 552 |
| | PLG | 8 | 1598 |
| | TIMP1 | 2 | 43 |
| | TIMP2 | 0 | 38 |
| | VTN | 0 | 485 |
| | FN1 | 6 | 931 |
| | FBN1 | 0 | 14 |
| | PRTN3 | 0 | 3 |
| | CTSK | 0 | 6 |
| | CTSL1 | 0 | 38 |
| | CTSL2 | 0 | 1 |
| | CTSS | 0 | 18 |
| Cytokines | IL5 | 4 | 29 |
| | IL6 | 3 | 980 |
| | IL8 | 3 | 1181 |
| | IL17 | 2 | 111 |
| | IL18 | 2 | 72 |
| | IL20 | 1 | 9 |
| | IL27 | 1 | 16 |
| | IL33 | 1 | 8 |
| | TNFA | 10 | 2143 |
| | INFA1 | 1 | 626 |
| | INFB1 | 2 | 168 |
| | INFG | 4 | 455 |
| | CHGA | 1 | 2 |
| | CCL4 | 0 | 34 |
| | LEP | 3 | 278 |
| Immune | TLR1 | 3 | 68 |
| Receptors | TLR2 | 2 | 21 |
| | TLR4 | 3 | 36 |
| | TLR6 | 2 | 1 |
| | IL6R | 1 | 137 |
| | CXCR1 | 1 | 56 |
| | CXCR2 | 2 | 131 |
| | IL18R1 | 1 | 7 |
| | IL20RA | 1 | 3 |
| | IL20RB | 1 | 1 |
| | IL27RA | 0 | 4 |
| | IL1RL1 | 0 | 2 |
| Natural | KLRG1 | 2 | 0 |
| Killer | A2M | | |
| Complex | CD69 | | |
| | CLEC1B | | |
| | CLEC7A | | |
| | KLRD1 | | |
| | NKG2D | | |
| | NKG2C | | |
| | NKG2A | | |
| | LY49L | | |
| Growth | PDGFA | 0 | 9 |
| Factors | PDGFB | 19 | 314 |
| | FGF1 | 6 | 257 |
| | FGF2 | 61 | 2918 |
| | FGF4 | 4 | 44 |
| | FGF5 | 1 | 12 |
| | FGF6 | 1 | 5 |
| | FGF11 | 2 | 131 |
| | TGFB1 | 12 | 740 |
| | TGFB2 | 1 | 93 |
| | EFNB2 | 8 | 114 |
| | ANGPT2 | 5 | 808 |
| Growth | FLT1 | 20 | 1555 |
| Factor | FGFR1 | 2 | 214 |
| Receptors | FGFR2 | 0 | 90 |
| | FGFR3 | 0 | 32 |
| | FGFR4 | 0 | 17 |
| | PDGFRA | 3 | 196 |
| | PDGFRB | 5 | 323 |
| | PPHB4 | 3 | 103 |
| | TIE2 | 6 | 772 |
| | TGFBR1 | 6 | 7 |
| | TGFBR2 | 4 | 11 |
| | TGFBR3 | 3 | 14 |
| Signaling | MAPK1 | 11 | 944 |
| Molecules | MAPK3 | 7 | 444 |
| | MAPK7 | 0 | 1 |
| | MAPK8 | 3 | 38 |
| | MAPK9 | 0 | 4 |
| | MAPK10 | 0 | 1 |
| | MAPK14 | 0 | 11 |
| | PTPN6 | 0 | 32 |
| | PTPN11 | 0 | 20 |
| | ABRA | 3 | 0 |
| | TMSB4X | 5 | 89 |
| | DLL1 | 5 | 53 |
| Transcription | JUN | 4 | 244 |
| Factors | FOS | 1 | 143 |
| | MYC | 2 | 356 |
| | EGR1 | 6 | 102 |
| | NFKB1 | 4 | 535 |
| | NFKB2 | 2 | 467 |
| | KAT2B | 1 | 2 |
| | NR4A1 | 0 | 7 |
| | HDAC5 | 0 | 7 |
| | ANKRD1 | 3 | 6 |
| Inhibitors | CD180 | manuscript | manuscript |
| | | in preparation | in preparation |
| | CDKN1A | 1 | 1 |

To confirm the relevance of our findings, we selected an additional 70 genes that we found upregulated, using microArray analysis, at the site of active arteriogenesis, the adductor muscle group, in C57Bl/6 mice subjected to single ligation of the left femoral artery (Table S2). We repeated the RTP for these 70 genes and looked for similarities in identified miRs between the two reverse target predictions (for 14q32 miRs, Table S3).

TABLE S2
Reverse Target Prediction 2; Target genes selected
from microArray data.
Target Gene

CCL19
CCL21
CCR7
FCER1G
MEF2A
MEF2B
MEF2C
MEF2D
USP18
IRF9
IRF1
IFIT2
PML
CCL5
VCAN
MMP3
SELL
CD44
LGALS3
CXCL13
CXCL10
PLCG2
VAV1
ARF6
TGFBR2
STAT3
RORC
LBP
MYD88
FCGR3A
ARPC1A
ARPC1B
ARPC2
ARPC3
ARPC4
ARPC5
IGFBP4
MSN
LCN2
DNAJB1
DBP
LRG1
CHI3L3
HSPA1B
S100A8
HSPA1A
C1QB
RRAD
S100A9
RNF213
MT2
SOCS3
PRG4
OASL2
SLPI
PHF11
MPEG1
CFB
KBTBD5
FPR2
SAP30
CEBPB
GADD45A
BST2
SLC6A9
C1QC
CXCL1
TUBA6
HSP105
IL1B

TABLE S3
14q32 miRs in RTPs 1 and 2.

| | Putative targets in RTP1 | Putative targets in RTP2 |
|---|---|---|
| 14q32 MicroRNA | (out of 127) | (out of 70) |
| 495 | 44 | 7 |
| 494 | 31 | 8 |
| 485 | 31 | 11 |
| 299 | 30 | 2 |
| 300 | 29 | 6 |
| 544 | 29 | 9 |
| 543 | 27 | 5 |
| 370 | 25 | 7 |
| 539 | 24 | 8 |
| 377 | 21 | 7 |
| 136 | 20 | 7 |
| 410 | 20 | 8 |
| 329 | 20 | 10 |
| 431 | 19 | 4 |
| 433 | 18 | 6 |
| 874 | 18 | 3 |
| 134 | 17 | 4 |
| 376c | 17 | 5 |
| 411 | 16 | 4 |
| 376a | 15 | 3 |
| 376b | 15 | 3 |
| 382 | 13 | 2 |
| 379 | 12 | 4 |
| 758 | 12 | 6 |
| 496 | 11 | 2 |
| 154 | 9 | 3 |
| 127 | 2 | 0 |

Both RTPs were performed looking for miR binding sites in human target genes to ensure clinical relevance. Conservation between human and murine target sites was checked to confirm the validity of our murine model of hind limb ischemia (for 14q32miRs, Table S4).

TABLE S4
Conservation of binding sites for miR-329,
miR-494 and miR-495 between mice and men.

| MiR-329 | | | MiR-494 | | | MiR-495 | | |
|---|---|---|---|---|---|---|---|---|
| Target | Sites in | Sites in | Target | Sites in | Sites in | Target | Sites in | Sites in |
| Gene | Humans | Mice | Gene | Humans | Mice | Gene | Humans | Mice |
| MEF2D | 4 | 1 | ARF6 | 3 | 2 | TGFBR1 | 3 | 2 |
| CXCR2 | 2 | 1 | TLR6 | 2 | 0 | STAT3 | 3 | 1 |
| TLR4 | 2 | 2 | EFNB2 | 2 | 1 | FAK | 2 | 1 |
| MEF2A | 2 | 2 | MYC | 2 | 0 | ITGAV | 2 | 3 |
| ITGB3 | 2 | 3 | ARPC5 | 2 | 1 | ITGB3 | 2 | 1 |
| FOS | 1 | 0 | MEF2D | 2 | 1 | CX43 | 2 | 1 |
| PTPN11 | 1 | 2 | RNF213 | 2 | 0 | CX45 | 2 | 1 |
| MAPK1 | 1 | 1 | EDN1 | 1 | 0 | ICAM1 | 2 | 0 |
| PDGFRA | 1 | 0 | VEGFA | 1 | 0 | CTSS | 2 | 0 |
| TGFBR1 | 1 | 0 | ITGB1 | 1 | 0 | TLR4 | 2 | 0 |
| FGF4 | 1 | 0 | ITGAL | 1 | 0 | IL8RB | 2 | 1 |
| PDGFB | 1 | 0 | VCAM1 | 1 | 0 | IL1RL1 | 2 | 0 |
| FGF5 | 1 | 0 | PECAM1 | 1 | 0 | KLRD1 | 2 | 0 |
| KLRG1 | 1 | 0 | SELP | 1 | 2 | PDGFA | 2 | 1 |
| CLEC7A | 1 | 2 | CCR2 | 1 | 1 | FGF5 | 2 | 0 |
| LEP | 1 | 0 | SERPIN1 | 1 | 0 | PTPN11 | 2 | 0 |
| PLAU | 1 | 1 | CTSS | 1 | 0 | JUN | 2 | 1 |
| PLAT | 1 | 0 | IL6 | 1 | 0 | HDAC4 | 2 | 0 |
| CTSS | 1 | 0 | IL33 | 1 | 1 | MEF2C | 2 | 3 |
| ITGAM | 1 | 1 | TNFA | 1 | 0 | EDN1 | 1 | 0 |
| CX45 | 1 | 1 | TLR4 | 1 | 1 | VEGFA | 1 | 1 |
| VEGFA | 1 | 1 | IL18R1 | 1 | 0 | VCAM1 | 1 | 0 |
| CCL19 | 1 | 0 | CLEC7A | 1 | 0 | CCL2 | 1 | 1 |
| IGFBP4 | 1 | 0 | KLRD1 | 1 | 0 | CDH5 | 1 | 0 |
| IRF1 | 1 | 0 | PDGFA | 1 | 0 | SERPINE1 | 1 | 1 |
| MEF2B | 1 | 0 | FGF2 | 1 | 1 | PLAT | 1 | 1 |
| MEF2C | 1 | 2 | FGF5 | 1 | 0 | PLG | 1 | 1 |
| MYD88 | 1 | 0 | FGFR2 | 1 | 3 | TIMP2 | 1 | 1 |
| PLCG2 | 1 | 0 | PDGFRA | 1 | 0 | IL5 | 1 | 0 |
| CEBPB | 1 | 0 | PPHB4 | 1 | 0 | IL33 | 1 | 0 |
| DNAJB1 | 0 | 1 | MAPK1 | 1 | 2 | INFG | 1 | 0 |
| SOCS3 | 0 | 0 | MAPK9 | 1 | 0 | LEP | 1 | 0 |
| EFNB2 | 1 | 2 | MAPK14 | 1 | 0 | IL6R | 1 | 0 |
| FGFR2 | 0 | 1 | PTPN11 | 1 | 0 | IL20RB | 1 | 0 |
| | | | JUN | 1 | 0 | KLRG1 | 1 | 0 |
| | | | KAT2B | 1 | 0 | CD69 | 1 | 0 |
| | | | ARPC1B | 1 | 1 | CLEC7A | 1 | 0 |
| | | | CD44 | 1 | 1 | FGF2 | 1 | 2 |
| | | | STAT3 | 1 | 1 | TGFB2 | 1 | 3 |
| | | | DNAJB1 | 1 | 1 | VEGFR | 1 | 0 |
| | | | MPEG1 | 0 | 1 | FGFR3 | 1 | 2 |
| | | | SLC6A9 | 0 | 1 | MAPK10 | 1 | 1 |
| | | | | | | DLL1 | 1 | 1 |
| | | | | | | KAT2B | 1 | 1 |
| | | | | | | HDAC5 | 1 | 0 |
| | | | | | | ARF6 | 1 | 0 |
| | | | | | | ARPC1A | 1 | 1 |
| | | | | | | CD44 | 1 | 1 |
| | | | | | | IFIT2 | 1 | 0 |
| | | | | | | MPEG1 | 1 | 0 |
| | | | | | | SOCS3 | 1 | 0 |
| | | | | | | RRAD | 0 | 1 |

### MicroArray

Healthy male adult C57Bl/6 mice were subjected to single ligation of the femoral artery, as described below. Four mice were sacrificed at each of the following time points: before ligation; 24 hours after ligation; 72 hours after ligation; 1 week after ligation. The adductor muscles were collected and snap-frozen on dry ice. Total RNA was isolated using the RNeasy fibrous tissue minikit (Qiagen). RNA concentration, purity and integrity were examined by nanodrop (NANODROP® Technologies) and Bioanalyzer (Agilent 2100) measurements.

For whole-genome expression profiling, amplified biotinylated RNA was generated using the Illumina TotalPrep RNA Amplification Kit. For array analysis, MouseWG-6 v2.0 Expression Beadchips (Illumina), which contain more than 45,200 transcripts, were used. Expression levels were Log 2-transformed and after quantile normalization, transcripts showing background intensity, both at baseline and after induction of HLI, were removed from the analysis.

MiR expression profiling was performed as two-color common reference hybridizations on LNA based arrays (MIRCURY LNA™ miR Array ready-to-spot probe set, Exiqon, Denmark), spotted in-house on CODELINK™ HD Activated slides (DHD1-0023, SurModics, Eden Prairie, Minn.) according to manufacturer's protocol. Samples were labeled with Hy5, by use of miRCURY LNA miR Array Power labeling kit (208032-A, Exiqon) and hybridized for 16 hours. Slides were washed (208021, Exiqon), scanned on an Agilent (G2565CA) Microarray scanner and analyzed by the Genepix 6.0 software. Normalization and background correction was performed in the “statistical language R” using “vsn” package (Bioconductor), and quadruplicate spots were averaged. Differential expression was assayed using the “limma” package (Bioconductor) by fitting the eBayes linear model and contrasting individual treatments with untreated controls. Log 2 fold changes were calculated using the toptable function of the limma package.

Analyses of mRNA, miR expression via rt/qPCR and expression of 14q32 miRs in various murine tissues is shown in FIG. 9.

### MicroRNA Inhibitors

AntagomiRs were designed with perfect reverse complementarity to the mature target miR sequence and purchased from VBC Biotech (Vienna, Austria). AntagomiRs were made up of a single-stranded O-methyl-modified RNA strand with 5′-end and 3′-end phosporothioate linkages and a 3′-end cholesterol tail.

Gene Silencing Oligonucleotides (GSOs) were designed with perfect reverse complementarity to the mature target miR sequence and synthesized at Idera Pharmaceuticals (Cambridge, Mass., USA)²¹. As a negative control, a scrambled sequence was used, designed not to target any known murine miR. GSOs were made up of two single-stranded O-methyl-modified DNA strands, linked together at their 5′ ends by a phosphorothioate-linker. Shielding the 5′-end of the single-stranded oligonucleotides prevents activation of the innate immune system via Toll-like Receptors; the double DNA strand increases specificity for the target miR.

Sequences of all antagomiRs and GSOs used are given in Table S5.

TABLE S5
Sequences of miRs, antagomiRs and GS0s.

| MiR | Sequence |
|---|---|
| hsa/mmu-miR-487b | 5′-AAUCGUACAGGGUCAUCCACUU-3′ |
| hsa/mmu-miR-494 | 5′-UGAAACAUACACGGGAAACCUC-3′ |
| hsa/mmu-miR-495 | 5′-AAACAAACAUGGUGCACUUCUU-3′ |
| mmu-miR-329 | 5′-AACACACCCAGCUAACCUUUUU-3′ |
| hsa -miR-329 | 5′-AACACACCUGGUUAACCUCUUU-3′ |
| GSO | Sequence |
| hsa/mmu-GSO-487b | 3′-TTAGCATGTCCCAGTAGGTGAA-X- |
| | AAGTGGATGACCCTGTACGATT-3′ |
| hsa/mmu-GSO-494 | 3′-ACTTTGTATGTGCCCTTTGGAG-X- |
| | GAGGTTTCCCGTGTATGTTTCA-3′ |
| hsa/mmu-GSO-495 | 3′-TTTGTTTGTACCACGTGAAGAA-X- |
| | AAGAAGTGCACCATGTTTGTTT-3′ |
| mmu-GSO-329 | 3′-TTGTGTGGGTCGATTGGAAAAA-X- |
| | AAAAAGGTTAGCTGGGTGTGTT-3′ |
| hsa-GSO-329 | 3′-TTGTGTGGACCAATTGGAGAAA-X- |
| | AAAGAGGTTAACCAGGTGTGTT-3′ |
| negative control GSO | 3′-TGTACGACTCCATAACGGT-X-TGGCAATACCTCAGCATGT-3′ |
| AntagomiR | Sequence |
| hsa/mmu-antagomiR-487b | 5′-AsAsGUGGAUGACCCUGUACGsAsUsUs-Chol-3′ |
| hsa/mmu-antagomiR-494 | 5′-GsAsGGUUUCCCGUGUAUGUUsUsCsAs-Chol-3′ |
| hsa/mmu-antagomiR-495 | 5′-AsAsGAAGUGCACCAUGUUUGsUsUsUs-Chol-3′ |
| mmu-antagomiR-329 | 5′-AsAsAAAGGUUAGCUGGGUGUsGsUsUs-Chol-3′ |
| hsa-antagomiR-329 | 5′-AsAsAGAGGUUAACCAGGUGUsGsUsUs-Chol-3′ |
| negative control antagomiR | 5′-AsUsGACUAUCGCUAUUCGCsAsUsGs-Chol-3′ |

′X′: Phosphorothioate linker
′-NNN-′: 2′-O-methyl-modified nucleotides
′s′: Phosphorothioate linkage
′Chol′: cholesterol group linked through a hydroxyprolinol-linkage

### Hind Limb Ischemia Models

Healthy adult male C57Bl/6 mice, aged 8 to 12 weeks (Charles River and Harlan) were housed in groups of 4 or 5 mice with free access to tap water and regular chow. All experiments were approved by the committee on animal welfare of the Leiden University Medical Center (Leiden, The Netherlands).

For miR-inhibition experiments, mice were given a bolus injection of 1 mg (˜40 mg/kg) GSO in PBS or PBS alone, 1 day prior to femoral artery ligation.

Mice were anesthetized by intraperitoneal (i.p.) injection of midazolam (8 mg/kg, Roche Diagnostics), medetomidine (0.4 mg/kg, Orion) and fentanyl (0.08 mg/kg, Janssen Pharmaceuticals). Unilateral hind limb ischemia was induced by electrocoagulation of the left femoral artery proximal to the superficial epigastric arteries alone (single ligation: model for effective arteriogenesis), or combined with electrocoagulation of the distal femoral artery proximal to the bifurcation of the popliteal and saphenous artery²² (double ligation: model for severe Peripheral Arterial Disease). After surgery, anesthesia was antagonized with flumazenil (0.7 mg/kg, Fresenius Kabi), atipamezole (3.3 mg/kg, Orion) and buprenorphine (0.2 mg/kg, MSD Animal Health).

Blood flow recovery to the paw was measured over time using Laser Doppler Perfusion Imaging (LDPI) as described.

Analgesic fentanyl (0.08 mg/kg) was administered subcutaneously after the final LDPI measurement and mice were sacrificed. The adductor, gastrocnemicus and soleus muscles were harvested and either snap-frozen on dry ice or fixed in 4% PFA.

Tissues were used for total RNA isolation for rt/qPCR analyses of miR and target gene expression or for immunohistochemistry, as described.

### Proliferation Assay in Primary Human Arterial Cells

Isolation and culture of primary human umbilical cord arterial endothelial cells (HUAECs), smooth muscle cells (HUASMCs) and fibroblasts (HUAFs) are described below.

Cells were seeded in 48-wells plates at 2500 (HUAFs) or 5000 (HUASMCs and HUAECs) cells per well. The next day, cells were incubated with GSOs (10 ng/μl for HUAFS and HUASMCs and 15 ng/μl for HUAECs) in culture medium. After 24 hours, medium was replaced by medium containing 0.5% FCS for HUAFs and HUASMCs or 10% NBCS for HUAECs with GSOs. Again after 24 hours, cells were stimulated for 24 hours for HUAFs and HUASMCs or 40 hours for HUAECs and consecutively, cell proliferation was determined by adding ³H-thymidine (PerkinElmer, Zaventum, Belgium) at a final concentration of 0.5 μCi/ml. After 5 hours, cells were washed with ice-cold PBS, fixed with 100% methanol, permeated with 5% tri-chloric acid and lysed with 0.3N NaCl. Disintegrations per minute (DPM) were counted for 5 minutes per sample in Ultima Gold™ scintillation cocktail (Canberra-Packard, Frankfurt, Germany).

rt/qPCR

MiR RT/qPCR

Total RNA was isolated using a standard TRIzol-chloroform extraction protocol. RNA concentration, purity and integrity were examined by nanodrop (NANODROP® Technologies). MiR quantification was performed according to manufacturer's protocol using TAQMAN® miR assays (Applied Biosystems). qPCRs were run on a 7900HT Fast Real-Time PCR System (Applied Biosystems), and amplification efficiencies were checked by standard curves. Normalization of data was performed using a stably expressed endogenous control (mmu-let-7c and mmu-miR-122 for in murine samples and hsa-miR-191 for human cell cultures).

mRNA RT/qPCR.

Relative quantitative mRNA PCR was performed on reverse transcribed cDNA using Taqman gene expression assays. qPCRs were run on a 7900HT Fast Real-Time PCR System (Applied Biosystems), and amplification efficiencies were checked by standard curves. Normalization of data was performed using stably expressed endogenous controls (GAPDH, HPRT1).

### Laser Doppler Perfusion Imaging

Blood flow recovery to the paw was measured over time using Laser Doppler Perfusion Imaging (LDPI) (Moore Instruments). Mice were anaesthetized by i.p. injection of midazolam (8 mg/kg) and medetomidine (0.4 mg/kg). Mice were place in a double-glazed pot, perfused with water at 37° C. for 5 minutes prior to each measurement. After LDPI, anesthesia was antagonized by subcutaneous injection of flumazenil (0.7 mg/kg) and atipamezole (3.3 mg/kg). LDPI measurements in the treated paw were normalized to measurements of the untreated paw, as internal control.

### Immunohistochemistry

α-SMA.

Five μm thick paraffin-embedded cross-sections of adductor muscle were re-hydrated and endogenous peroxidase activity was blocked. Smooth muscle cells were stained with anti-α-smooth muscle actin (anti-α-SMA) (DAKO, Glostrup, Denmark). Sections were counterstained with hematoxylin. α-SMA positive arterioles were analyzed using image analysis (Image J 1.43, NHI, USA).

CD31.

In 6 μm thick fresh-frozen cross-sections of soleus muscle, endothelial cells were fixed in ice-cold acetone and stained with anti-CD31 (BD Pharmingen). Sections were counterstained with hematoxylin. Quantification of CD31 positive area was performed on sections photographed randomly (six representative images per muscle per mouse, three animals per group) using image analysis (Qwin, Leica, Wetzlar, Germany).

### Isolation of Human Umbilical Arterial Endothelial Cells, Smooth Muscle Cells and Fibroblasts (HUAECs, HUASMCs & HUAFs)

Umbilical cords were collected from full-term pregnancies and stored in sterile PBS at 4° C. and subsequently used for cell isolation within 7 days. For HUAEC isolation, a cannula was inserted in one of the umbilical arteries and flushed with sterile PBS. The artery was infused with 0.075% collagenase type II (Worthington, Lakewood, N.J., USA) and incubated at 37° C. for 20 minutes. The collagenase solution was collected and the artery was flushed with PBS in order to collect all detached endothelial cells. The cell suspension was centrifuged at 400 g for 5 minutes and the pellet was resuspended in HUAEC culture medium (M199 (PAA, Pasching, Austria), 10% heat inactivated human serum (PAA), 10% heat inactivated newborn calf serum (PAA), 1% penicillin/streptomycin (MP Biomedicals, Solon, Ohio, USA), 150 μg/ml endothelial cell growth factor (kindly provided by Dr. Koolwijk, VU Medical Center, Amsterdam, the Netherlands) and 0.1% heparin (LEO Pharma, Ballerup Danmark). HUAECs were cultured in plates coated with 1% gelatin.

The second artery was removed and cleaned from remaining connective tissue. Endothelial cells were removed by gently rolling the artery over a blunted needle. The tunica adventitia and tunica media were separated using surgical forceps. After overnight incubation in HUASMC/HUAF culture medium, (DMEM GLUTAMAX™ (Invitrogen, GIBCO, Auckland, New Zealand), 10% heat inactivated fetal bovine serum (PAA), 10% heat inactivated human serum, 1% penicillin/streptomycin and 1% nonessential amino acids (PAA)), both tunicae were incubated separately in a 2 mg/ml collagenase type II solution (Worthington) at 37° C. Cell suspensions were filtered over a 70 μm cell strainer and centrifuged at 400 g for 10 minutes. Cell pellets were resuspended and plated in culture medium. Cells isolated from the tunica adventitia were washed with culture medium after 90 minutes to remove slow-adhering non-fibroblast cells.

### Primary Cell Culture

Cells were cultured at 37° C. in a humidified 5% CO₂ environment. Culture medium was refreshed every 2-3 days. Cells were passed using trypsin-EDTA (Sigma, Steinheim, Germany) at 90-100% (HUAECs and HUASMCs) or 70-80% confluency (HUAFs). HUASMCs and HUAFs were used up to passage six and HUAECs up to passage three. Stock solutions of isolated HUASMCs and HUAFs up to passage four and HUAECs up to passage two were stored at −180° C. in DMEM GLUTAMAX™ containing 20% FBS and 10% DMSO (Sigma).

### Results

### Reverse Target Prediction

We performed two reverse target prediction analyses. In the initial RTP, we included 127 genes that are known to be involved in neovascularization from both literature and our own studies (Table 51). As anticipated, we observed enrichment of putative binding sites for several miRs that were previously reported to influence post-ischemic neovascularization, including miR-17/92a¹⁰ (29 and 21 putative target genes, respectively), miR-106b/93/25^{8, 9}(29, 29 and 21 putative target genes respectively), the miR-15a family^{11, 12, 17} (26 putative target genes), miR-503¹⁴ (11 putative target genes) and miR-100⁷ (2 putative target genes) but not, for example, miR-126⁶. However, more outspoken was the enrichment of putative binding sites for 27 miRs from the 14q32 miR gene cluster, including miR-329, miR-494 and miR-495 (20, 31 and 44 putative target genes, respectively) (Table S3).

In the second RTP, we included 70 additional genes that we found upregulated in the early stages of vascular remodeling in murine tissue after hind limb ischemia induced by single ligation of the femoral artery, a model for effective neovascularization (Table S2). We again observed enrichment of binding sites for miR-17/92 (8 and 4 putative target genes, respectively), miR-106/93/25 (8, 8 and 4 putative target genes respectively), the miR-15a family (3 putative target genes), miR-503 (5 putative target genes) and miR-100 (1 putative target genes). Furthermore, enrichment of binding sites for 26 of the initial 27 identified 14q32 miRs was recovered, including miR-329, miR-494 and miR-495 (10, 8 and 7 putative target genes, respectively) (Table S3).

We used human target gene sequences in both RTPs and checked for conservation between human and murine target sites. The 14q32 miR gene cluster is highly conserved between mammals and we found that many, but not all, putative target sites are conserved as well (Table S4).

### MicroArray

We performed microarray analyses of miR expression profiles on the same murine tissue samples used for mRNA microarray analyses. At 24 and 72 hours after hind limb ischemia (single femoral artery ligation), 14q32 miR-494 was the most significantly regulated microRNA. MiR-494 was rapidly downregulated at 24 hours and at 72 hours; expression normalized after 7 days (FIG. 2). When we looked at other 14q32 miRs, we observed that more than half the cluster members were downregulated during effective neovascularization. Ten 14q32 miRs were already downregulated 24 hours after ischemia (early-responders); 24 14q32 miRs were first downregulated 72 hours after ischemia (late responders); sixteen 14q32 miRs were not regulated at all (non-responders); nine 14q32 miRs were not on the microArray (Table S6).

TABLE S6
Murine 12F1 miR downregulation during effective neovascularization.

| Early Responder | Late Responder | Non-Responder | Not on Array |
|---|---|---|---|
| miR-337 | miR-770 | miR-493 | miR-1906-1 |
| miR-432 | miR-673 | miR-540 | miR-3070a |
| miR-494 | miR-665 | miR-431 | miR-3070b |
| miR-666 | miR-433 | miR-136 | miR-1188 |
| miR-487b | miR-127 | miR-370 | miR-3071 |
| miR-134 | miR-434 | miR-379 | miR-1197 |
| miR-453 | miR-341 | miR-380 | miR-1193 |
| miR-154 | miR-882 | miR-758 | miR-3072 |
| miR-409 | miR-411 | miR-543 | miR-1247 |
| miR-410 | miR-299 | miR-495 | |
| | miR-323 | miR-376c | |
| | miR-329 | miR-539 | |
| | miR-679 | miR-544 | |
| | miR-667 | miR-485 | |
| | miR-654 | miR-496 | |
| | miR-376b | miR-541 | |
| | miR-376a | | |
| | miR-300 | | |
| | miR-381 | | |
| | miR-382 | | |
| | miR-668 | | |
| | miR-377 | | |
| | miR-412 | | |
| | miR-369 | | |

For further studies, we choose to investigate one 14q32 miR from each responder group, that all were predicted to target multiple neovascularization genes. We selected miR-494 (early-responder), miR-329 (late responder) and miR-495 (non-responder). MiR-487b was the second most significantly downregulated 14q32 miR (early-responder). Although it was not identified in either RTP, we did previously report that miR-487b plays an important role in outward remodeling of the aorta²⁴ and therefore we also included miR-487b in our further studies.

### Gene Silencing Oligonucleotides

We compared the efficacy and specificity of GSOs to the more commonly used antagomiRs, using cultures of primary human arterial adventitial fibroblasts. Both antagomiRs and GSOs proved equally potent in inhibiting expression of their target miR in HUAFs (FIGS. 3A & B). However, when we measured expression of other miRs, we found that where GSOs inhibited only the expression of their target miR, antagomiRs influenced expression of other miRs as well (FIG. 3C). GSOs therefore showed equal efficacy to, but higher specificity than, antagomiRs in primary arterial fibroblasts.

### In Vivo MiR Inhibition

We used rt/qPCR to confirm down-regulation of the targeted 14q32 miRs in the adductor muscles of GSO-treated mice. All four GSOs achieved significant knockdown of their target miR at days 3 and 7 after surgery, corresponding to days 4 and 8 after injection, compared to the GSO-control (p-value for GSO-494 at day 7 was 0.06). At day 17 after surgery, day 18 after GSO injection, expression of miR-329, miR-494 and miR-495 had normalized, but miR-487b was still downregulated (FIG. 3D).

### Blood Flow Recovery In Vivo

Mice were given a bolus injection of 1 mg GSO in PBS, or PBS alone, via the tail vein. The next day, they were subjected to double ligation of the left femoral artery, a model for severe peripheral artery disease. Blood flow recovery to the paw was followed by LDPI up to 17 days after hind limb ischemia. Mice in all groups appeared healthy and no significant weight loss was observed. All four treatment groups, GSO-329, GSO-487b, GSO-494 and GSO-495, showed drastically improved blood flow recovery compared to both the PBS and the GSO-control groups (FIG. 4A). There were no significant differences between the PBS and GSO-control groups. Mice that received either GSO-329 or GSO-495 showed an increase in perfusion compared to GSO-control as early as 3 days after induction of ischemia. The increase in perfusion persisted over time in both groups and mice treated with GSO-329 even made a full recovery in paw perfusion within an astounding seven days after induction of ischemia, compared to approximately 60% recovery in GSO-control treated mice (FIG. 4B). Mice treated with GSO-495 or GSO-494 had nearly fully recovered perfusion after ten days followed by mice treated with GSO-487b at two weeks. The GSO-control and PBS groups did not make full recoveries before being sacrificed at day seventeen.

### In Vivo Arteriogenesis

We determined the number and diameter of arterioles by analyzing α-SMA positive vessels in the adductor muscles of GSO-treated mice sacrificed 7 days after double ligation of the left femoral artery. Collateral arteries form from pre-existing arterioles. Therefore, we were not surprised that the number of collateral arteries between the left and right paw either within groups, or between groups, were similar. However, we did observe increases in arteriole diameters between the left and right adductor muscles compared to the GSO control for mice treated with GSO-329 (2.6-fold, p=0.09) and GSO-487b (3-fold, p=0.02). Arteriole diameters also appeared increased in mice treated with GSO-494 (1.9-fold, p=0.3) and GSO-495 (2.3 fold, p=0.3), indicating GSO-induced increases in arteriogenesis (FIG. 5).

### In Vivo Angiogenesis

The left (ischemic) and right (normoxic) soleus muscles of GSO-treated mice, sacrificed 7 days after double ligation of the left femoral artery, were stained for CD31 to visualize capillary formation. Increases in capillary formation compared to mice treated with GSO control were observed in the left solei of mice treated with GSO-329 (9.5-fold, p=0.003) and GSO-494 (8.1-fold, p=0.06). Capillary formation also appeared increased in the left solei of mice treated with GSO-487b (4.2-fold, p=0.1) and GSO-495 (5.9-fold, p=0.2)(FIG. 6).

### In Vivo Target Gene Regulation

We selected the 14q32 microRNAs for their potential to target a broad range of pro-arteriogenic and pro-angiogenic target genes. As it would not be feasible to confirm regulation of all putative targets, we made a selection based on the number of target sites, conserved between humans and mice, in the 3′UTRs of potential 14q32 miR targets (Table S5). For miR-329, we selected TLR4, MEF2A, ITGB3, EFNB2, VEGFA and FGFR2; for miR-494, we selected ARF6 TLR4, VEGFA, EFNB2 and FGFR2; for miR-495, we selected TGFB2, ITGAV, STAT3 and TGFBR. As we have previously shown, miR-487b has only 14 conserved putative target genes. We confirmed that miR-487b directly targets the vasoactive Insulin Receptor Substrate 1 (IRS1) in the arterial wall, leading to increased survival of both medial smooth muscle cells and adventitial fibroblasts²⁴.

We used rt/qPCR to determine whether these genes were upregulated in the left adductor muscles of mice treated with the relevant GSOs. In the adductor muscles of mice treated with GSO-329, we observed up-regulation of several target genes for miR-329, including TLR4, ITGB3, VEGFA and FGFR2 at 3, but not at 7 days after HLI (FIG. 7A-B). In mice treated with GSO-494, we observed upregulation of target genes TLR4 and VEGFA at day 3 and of TLR4, ARF6 and FGFR2 at day 7 after HLI (FIG. 7C-D). As miR-329 was a late-responder, the strongest benefits of miR-329 inhibition were expected to be observed early after HLI, in contrast to miR-494 which as an early-responder was downregulated rapidly after HLI and therefore benefits of additional inhibition were expected to be observed at later time points. Even though miR-495 was efficiently downregulated and we observed stimulatory effects of GSO-495 on neovascularization and blood flow recovery, we could not confirm upregulation of putative target genes in mice treated with GSO-495 via rt/qPCR (FIG. 10).

### In Vitro Effects of 14q32 MiR Inhibition

Whereas angiogenesis depends mainly on activation and proliferation of endothelial cells alone, arteriogenesis requires activation and proliferation of arterial endothelial cells, smooth muscle cells and fibroblasts. Therefore, we studied the effect of GSO treatment on these three cell types. None of the GSOs had effects on proliferation of smooth muscle cells (FIG. 11), as we had previously shown for GSO-487b²⁴. In arterial endothelial cells however, inhibition of miR-329, miR-487b and miR-495 all led to increased cell proliferation by approximately 20%, 50% and 35% respectively. Inhibition of miR-494 did not affect endothelial cell proliferation (FIG. 8A). In contrast, in fibroblasts we observed an increase of 20% in cell proliferation after miR-494 inhibition (FIG. 8B), whereas no effects were observed for the other GSOs.

### Discussion

In this study, we show that inhibition of individual 14q32 miRs improves blood flow recovery and stimulates both arteriogenesis in the adductor muscle and angiogenesis in the ischemic calf muscle. We made use of their master switch character to identify miRs that regulate neovascularization via Reverse Target Prediction. In a total set of nearly 200 genes associated with both angiogenesis and arteriogenesis, there was enrichment for binding sites of 27 miRs that all belong to the miR gene cluster on human chromosome 14. 14q32 miRs are downregulated during effective neovascularization in mice. We used Gene Silencing Oligonucleotides to inhibit the expression of four 14q32 miRs in vivo and followed blood flow recovery to the paw after double ligation of the left femoral artery in mice.

Although previous studies have demonstrated beneficial effects of individual miR inhibition on post-ischemic neovascularization, the effect size observed here, particularly for miR-329, is unprecedented. In 2009, Bonauer et al¹⁰ reported on the role of the miR-17/92a cluster in angiogenesis. They showed that inhibition of miR-92a improved post-ischemic blood flow recovery in C57Bl/6 mice. Although the authors do not report the actual percentages of blood flow recovery, their LDPI images clearly show that mice have not recovered at day 14 after double ligation of the femoral artery and vein. In 2011, Grundmann et al⁷ showed that inhibition of miR-100 also leads to improved blood flow recovery after ischemia. The authors described an absolute increase in perfusion of 10 to 15% at day 7 after double ligation of the femoral artery in C57BL/6 mice. Finally in 2012, Yin et al¹¹ took an opposite approach and demonstrated that over-expression of miR-15a attenuates post-ischemic blood flow recovery, resulting in an absolute decrease of perfusion of 10% at day 7 and 25% at day 14 after excision of the femoral artery, compared to control animals.

Using a large-scale RTP to identify miRs that regulate neovascularization, or any physiological process for that matter, is a novel approach. For neovascularization, the RTP proved both a robust and effective method. We recovered most miRs previously reported to be associated with angiogenesis and identified a large set of novel neovascularization miRs, the 14q32 miR gene cluster. The fact that inhibition of 14q32 miRs indeed increases blood flow recovery after ischemia and enhanced both arteriogenesis and angiogenesis in vivo, supports the validity of this novel method.

Interestingly however, we did not only recover miRs previously reported to inhibit neovascularization, but also miRs previously reported to enhance neovascularization, including miR-106b/93/25^{8, 25} and miR-424²³, although miR-424 has also been reported to have anti-angiogenic effects²⁶. Our list of target genes consisted mainly of pro-arteriogenic and pro-angiogenic genes; miRs predicted to target these genes are therefore likely to inhibit neovascularization. Perhaps, regulation of pro-neovascularization and anti-neovascularization pathways are more tightly intertwined than previously thought. This finding also indicates that the RTP could be used to identify pro-arteriogenic miRs, targeting anti-arteriogenic genes. Neovascularization could potentially be improved by the use of miR-mimics, leading to over-expression of these miRs and down-regulation of their anti-arteriogenic targets. However, miR-overexpression by use of e.g. miR-mimics is likely to lead to more off-target effects than inhibition, as miR over-expression and hence gene-inhibitory activity in organs and tissues not endogenously expressing the targeted miR can likely occur.

Although several miR-target gene prediction algorithms are available online, we chose to restrict our RTPs to www.targetscan.org. In a previous study on polymorphisms in miR-binding sites, we found predictions made by TargetScan to have approximately 60% accuracy²⁷; combined with the large number of genes included in the RTP, TargetScan's predictions alone proved robust enough to identify important neovascularization miRs. MicroArray analyses showed downregulation of most 14q32 miR gene cluster members during effective neovascularization, which further validates the findings of both RTPs.

The 14q32 miR gene cluster is highly conserved between humans and mice. Of the four 14q32 miRs selected for in vivo silencing here, only the sequence of hsa-miR-329 varied slightly from its murine variant mmu-miR-329 (Table 51). Yet, many putative binding sites were conserved between humans and mice. Surprisingly, miR-495, which had the most putative pro-arterio and -angiogenic targets in RTP1, had the least conserved target sites of the four selected miRs. As conservation over species often reflects the biological significance of genomic sequences, perhaps this lower degree of conservation explains the more moderate effects of miR-495 inhibition on neovascularization as measured by immunohistochemistry. It may also explain why miR-495 was not regulated during effective vascular remodeling and neovascularization in mice, why it had less putative targets in the evidence-based RTP2 and why we could not confirm upregulation of putative target genes after GSO-495 treatment.

MiR-487b is an exceptional miR as it has only 14 conserved putative target genes in both humans and mice. We previously confirmed the role of miR-487b in outward remodeling of the aorta, via targeting the pro-survival factor Insulin Receptor Substrate 1 (IRS1) in vivo in rats and in vitro in human primary arterial cells²⁴. Having only a single conserved neovascularization target gene, IRS1, most likely explains the slightly more moderate effects of miR-487b inhibition on blood flow recovery and neovascularization compared to miR-329 and miR-494 inhibition. A recent study on the role of miR-487b in human lung cancer did confirm SUZ12, BMI1, WNT5A and KRAs as direct targets for miR-487b²⁸. However, except for one of two sites in the WNT5A 3′UTR, the binding sites for miR-487b in these genes were not conserved in mice and can therefore not have contributed to the effects on neovascularization in our murine model.

We set out to identify miRs that act as master switches, having perhaps only moderate effects on expression levels, but of many different target genes, involved in all aspects of arteriogenesis. Particularly miR-329 and miR-494 proved to regulate most of the selected target genes in vivo. These target genes, involved in various aspects of vascular remodeling were upregulated in vivo after miR-329 or miR-494 inhibition. Correspondingly, effects on blood flow recovery, arteriogenesis and angiogenesis were robust. Inhibition of miR-329 resulted in an unprecedented rapid recovery of paw perfusion. As miR-329 was late-responder in our microArray analyses, perhaps miR-329 inhibition in the early stages of neovascularization greatly enhances the process as a whole. This hypothesis is supported by the observation that endothelial cell proliferation was enhanced after treatment with GSO-329, as arteriogenesis is initiated after shear stress-induced endothelial activation. Like miR-329, inhibition of miR-495 also led to increased paw perfusion in the very early stages of post-ischemic neovascularization. Besides the here shown increased proliferation of endothelial cells after miR-495 inhibition, previous studies have also shown a role for miR-495 in both cell survival and migration²⁹⁻³¹. Inhibition of miR-487b also increases endothelial proliferation.

Our microarray analyses showed that expression of miR-494, after a quick downregulation, starts to normalize within a week after ischemia. Of the four GSOs used in this study, GSO-494 is the slowest starter with respect to blood flow recovery, but especially between days 7 and 10 after femoral artery ligation, GSO-494 treatment improves paw perfusion compared to the control. MiR-494 was previously reported to impact both proliferation and survival of, amongst other cell types, cardiac myocytes^{32, 33}. We observed that miR-494 did not impact arterial endothelial cell proliferation, but enhanced arterial adventitial fibroblast proliferation, which is in agreement with the slow start followed by stronger increases in flow, particularly in the later stages of neovascularization (i.e. fibroblast recruitment and reinstatement of the extracellular matrix), that we observed in vivo. Potentially, combined administration of GSO-329 and GSO-494 would therefore enhance post-ischemic blood flow recovery and neovascularization even further.

In conclusion, we here demonstrate that inhibition of individual 14q32 miRs leads to increases in post-ischemic blood flow recovery in vivo. We believe that 14q32 miRs function as master switches in vascular remodeling and neovascularization. Inhibition of either individual, or of a combination of, 14q32 miRs, may in the future offer an alternative to growth factors in therapeutic neovascularization.

### EXAMPLE 2

### Material and Methods

Reverse Target Prediction Based on existing knowledge from both literature^{xiv,xv,xvi,xvii,xviii} and previous studies within our group, we compiled a list of 163 genes involved in atherosclerosis, including chemokines, cytokines, adhesion molecules, scavenger receptors, lipid related targets, complement factors, matrix metalloproteinases (MIMPs) and growth factors. All genes were divided into two groups: one which was expected to reduce (43 genes) and one which was expected to aggravate (120 genes) atherosclerosis. Using the online algorithm Targetscan (www.targetscan.org), lists were generated of all miRs predicted to target our selected genes. These lists were then transferred to a spreadsheet and the listing number, the number of times an individual miR was listed in the total spreadsheet, was counted manually for each miR. We ranked the miRs according to their prevalence, which means that the miR with the highest occurrence of 3′UTR binding sites is predicted to regulate the most atherosclerosis-related genes (Table 1a). The RTP was performed by analyzing binding sites in human target genes to ensure clinical relevance. We checked the conservation between human and murine target sites afterwards, confirming the validity of the use of our mouse model for atherosclerosis.

TABLE 1a

| | # of predicted | | # of predicted |
|---|---|---|---|
| Top 30 Pro-atherogenenic miRs | genes | Top 30 Anti-atherogenenic miRs | genes |
| miR-590-3p | 48 | miR-590-3p | 17 |
| miR-24/24ab/24-3p | 36 | miR-186 | 13 |
| miR-129-5p/129ab-5p | 35 | miR-410/344de/344b-1-3p | 12 |
| miR-495/1192 | 35 | miR-543 | 12 |
| miR-340-5p | 35 | miR-384/384-3p | 12 |
| miR-150/5127 | 34 | miR-485-5p/1698/1703/1962 | 12 |
| miR-186 | 33 | miR-326/330/330-5p | 12 |
| miR-543 | 33 | miR-329/329ab/362-3p | 11 |
| miR-33ab/33-5p | 32 | miR-203 | 11 |
| miR-149 | 31 | miR-17/17-5p/20ab/20b-5p/93/106ab/427/518a- | 11 |
| | | 3p/519d | |
| miR-203 | 31 | miR-93/93a/105/106a/291a 3p/294/295/302abcde/ | 11 |
| | | 372/373/428/519a/520be/520acd-3p/1378/1420c | |
| miR-494 | 30 | miR-218/218a | 11 |
| miR-410/344de/344b-1-3p | 30 | miR-214/761/3619-5p | 11 |
| miR-290-5p/292-5p/371-5p/293 | 30 | miR-181abcd/4262 | 11 |
| miR-300/381/539-3p | 30 | miR-300/381/539-3p | 11 |
| miR-128/128ab | 30 | miR-539/539-5p | 11 |
| miR-326/330/330-5p | 29 | miR-129-5p/129ab-5p | 11 |
| miR-376c/741-5p | 28 | miR-320abcd/4429 | 11 |
| miR-204/204b/211 | 28 | miR15abc/16/16abc/195/322/424/497/1907 | 11 |
| miR-181abcd/4262 | 28 | miR-544/544ab/544-3p | 11 |
| miR-539/539-5p | 28 | miR-495/1192 | 10 |
| miR-124/124ab/506 | 27 | miR-19ab | 10 |
| miR-185/882/3473/4306/4644 | 27 | miR-145 | 10 |
| miR-9/9ab | 27 | miR-425/425-5p/489 | 10 |
| miR-197 | 27 | miR-374ab | 10 |
| miR-23abc/23b-3p | 26 | miR-125a-3p/1554 | 10 |
| miR-143/1721/4770 | 26 | miR-150/5127 | 10 |
| miR-874 | 26 | miR-23abc/23b-3p | 9 |
| miR-93,93a/105/106a/291a-3p/ | 25 | miR-376c/741-5p | 9 |
| 294/295/302abcde/372/373/428/519a/ | | | |
| 520be/520acd-3p/1378/1420ac | | | |
| miR-134/3118 | 25 | miR-200bc/429/548a | 9 |

### Mice

All animal work was performed in compliance with the Dutch government guidelines and the Directive 2010/63/EU of the European Parliament. Male apoE^{-/-} mice, obtained from the local animal breeding facility (Gorlaeus Laboratories, Leiden, the Netherlands), were fed a Western type diet, containing 0.25% cholesterol and 15% cacao butter (SDS, Sussex, UK) for six weeks. Before surgical intervention mice were age-, cholesterol-, and weight-matched. Details of cholesterol measurement are described below. White blood cell (WBC) numbers and cellular differentiation were determined on a Sysmex cell differentiation apparatus (Goffin Meyvis, Etten-Leur, The Netherlands).

### Surgical Intervention

Two weeks after start of the Western type diet, carotid artery plaque formation was induced by perivascular collar placement as described previously.^{xix} In brief, a semi-constrictive collar was placed around both left and right carotid arteries of the mice. Low shear stress and disturbed flow at the proximal site of the collar result in increased expression of endothelial adhesion molecules and atherosclerotic lesion formation. Four weeks after collar placement, mice were anaesthetized and in situ perfused, after which carotid artery lesions were analyzed.

Treatment with GSOs

At day 4 after surgery mice received an intravenous injection of either 1 mg Gene Silencing Oligonucleotide (GSO, kindly provided by Idera Pharmaceuticals, Cambridge, Mass., USA) or PBS control. A subset of mice (n=6 per group) was sacrificed 4 days later in order to establish down-regulation of miRs in vivo. For effects on atherosclerosis, the remaining mice received a second injection of 0.5 mg GSO per mouse at day 18 (n=15 per group). GSOs were designed with perfect reverse complementarity to the mature target miR sequence and synthesized by Idera Pharmaceuticals. As a negative control, a scrambled sequence was used, designed not to target any known murine miR. GSOs consist of two single-stranded O-methyl-modified DNA strands, linked together at their 5′ends by a phosphorothioate-linker to avoid TLR-activation. Sequences of all GSOs used are given in Table S1a.

TABLE S1a
GSO sequences

| MiR | Sequence |
|---|---|
| mmu-miR-494 | 5′-UGAAACAUACACGGGAAACCUC-3′ |
| mmu-miR-495 | 5′-AAACAAACAUGGUGCACUUCUU-3′ |
| mmu-miR-329 | 5′-AACACACCCAGCUAACCUUUUU-3′ |
| hsa-miR-329 | 5′-AACACACCUGGUUAACCUCUUU-3′ |
| GSO | Sequence |
| mmu-GSO-494 | 3′-ACTTTGTATGTGCCCTTTGGAG-X-GAGGTTTCCCGTGTATGTTTCA-3′ |
| mmu-GSO-495 | 3′-TTTGTTTGTACCACGTGAAGAA-X-AAGAAGTGCACCATGTTTGTTT-3′ |
| mmu-GSO-329 | 3′-TTGTGTGGGTCGATTGGAAAAA-X-AAAAAGGTTAGCTGGGTGTGTT-3′ |
| hsa-GSO-329 | 3′-TTGTGTGGACCAATTGGAGAAA-X-AAAGAGGTTAACCAGGTGTGTT-3′ |
| negative control | 3′-TGTACGACTCCATAACGGT-X-TGGCAATACCTCAGCATGT-3′ |
| GSO | |

### Cholesterol Measurement and Analysis

Blood was collected from the mice by tail bleeding. The concentration of cholesterol in plasma was determined by incubation with 0.025 U/ml cholesterol oxidase (Sigma) and 0.065 U/ml peroxidase and 15 μg/mL cholesterol esterase (Roche Diagnostics, Mannheim, Germany) in polyoxyethylene-9-laurylether, and 7.5% methanol). Precipath (standardized serum; Boehringer Mannheim, Germany) was used as an internal standard. Absorbance was measured at 490 nm.

For lipid profiling, plasma was pooled (n=3 mice per sample) and diluted 6 times, after which fractionation of plasma lipoproteins was performed using an AKTA-FPLC. Triglyceride levels and total cholesterol levels were determined in each fraction and in the original pooled sample by incubation with cholesterol CHOD-PAP Reagent (Roche/Hitachi). Absorbance was measured at 492 nm.

### Histology and Morphometry

Paraffin sections (5 μm thick) were routinely stained with HPS (hematoxylin-phloxine-saffron), which were used to determine plaque size. Picrosirius red staining was used to visualize collagen and for measurement of necrotic core size. Plaque composition was further examined by staining for smooth muscle cells (alpha smooth muscle actin, Sigma) and macrophages (MAC 3, BD-Pharmingen). The amount of mast cells and their activation status was visualized using an enzymatic staining kit (Naphtol-CAE, Sigma).

Morphometric analysis (Leica Qwin image analysis software) was performed on HPS-stained atherosclerotic lesions at site of maximal stenosis. (Immuno) histochemical stainings were quantified by computer assisted analysis (Leica, Qwin) and expressed as the percentage of positive stained area of the total lesion area. Mast cells were counted manually. A mast cell was considered resting when all granula were maintained inside the cell, while mast cells were assessed as activated when granula were deposited in the tissue surrounding the mast cell. The necrotic core was defined as the a-cellular, debris-rich plaque area as percentage of total plaque area.

### Cell Culture

BM (bone marrow) cells isolated from C57Bl/6 mice were cultured for 7 days in RPMI medium supplemented with 20% fetal calf serum (FCS), 2 mmol/L 1-glutamine, 100 U/mL penicillin and 100 μg/mL streptomycin and 30% L929 cell-conditioned medium, as the source of macrophage colony-stimulating factor (M-CSF), to generate BM-derived macrophages (BMDMs).^{xx} For generation of primary mast cells, BM cells were cultured in RPMI medium supplemented with 10% IL-3 supernatant (supernatant of WEHI-cells overexpressing and secreting murine Interleukin 3 (mIL3)), 1 mM sodium pyruvate, MEM non-essential amino acids, 10% FCS, 2 mmol/L 1-glutamine, 100 U/mL penicillin and 100 μg/mL streptomycin for 4 weeks.^{xxi} Mast cell purity and maturation was determined microscopically by staining of cytospins with 0.5% aqueous toluidin blue. Primary cultured murine smooth muscle cells (vSMC) and cell lines for fibroblasts (3T3) and endothelial cells (H5V) were cultured in complete DMEM medium supplemented with 10% FCS, 2 mmol/L 1-glutamine, 100 U/mL penicillin and 100 μg/mL streptomycin.

Mast cells, fibroblasts, smooth muscle cells and endothelial cells were plated in triplicate at a density of 10⁶ cells/mL. GSOs were added overnight at a concentration of 5 ng/mL, after which the cells were lysed for RNA isolation.

For BMDMs, GSOs were added immediately after isolation from BM in a concentration of 5 ng/mL. After three days medium was refreshed with a similar addition of GSOs in a concentration of 5 ng/mL. Four days later, medium was removed cells were lysed for RNA isolation.

RNA isolation, cDNA synthesis and qPCR

Three carotid artery segments from 7 days after collar placement were pooled and homogenized by grounding using a Pellet Pestle Cordless Motor (Kimble Chase Life Science, USA). Total RNA was isolated using a standard TRIzol-chloroform extraction protocol. RNA concentration, purity and integrity were examined by nanodrop (NANODROP® Technologies). MiR quantification was performed according to manufacturer's protocol using TaqMan® miR assays (Applied Biosystems, Foster City, Calif.). qPCRs were run on a 7900HT Fast Real-Time PCR System (Applied Biosystems). Normalization of data was performed using a stably expressed endogenous control (mmu-let-7c).

For the in vitro experiments, total RNA was extracted from the cells using the guanidine thiocyanate (GTC) method^{xxii}. RNA was reverse transcribed by M-MuLV reverse transcriptase (RevertAid, MBI Fermentas, Leon-Roth) and used for quantitative analysis of mouse genes (Table S2a) with an ABI PRISM 7700 Taqman apparatus (Applied Biosystems). Murine HPRT and RPL27 were used as standard housekeeping genes.

TABLE S2a
primers used for in vitro experiments

| Gene | Forward primer | Reversed primer |
|---|---|---|
| CXCR4 | GGTGATCCTGGTCATGGGTT | TGACAGGTGCAGCCGGTA |
| CDKN1B | CGGCTGGGTTAGCGGAGCAGTGT | CCAGCGTTCGGGGAACCGTCTGAA |
| TIMP2 | GTTTATCTACACGGCCCCCTCTT | ATCTTGCCATCTCCTTCTGCCTT |
| TIMP3 | ACTGTGCAACTTTGTGGAGAGGT | GAGACACTCATTCTTGGAGGTCA |
| CXCL12 | TGCATCAGTGACGGTAAACCA | GGCTCTCGAAGAACCGGC |
| ADIPOR2 | CATGTTTGCCACCCCTCAGTATC | AGCCAGCCTATCTGCCCTATG |
| IL10RA | GACGGCATCATCTATGGGACA | GACTTGTTCGTACTCATCCCCTG |
| IL10 | GGGTGAGAAGCTGAAGACCCTC | TGGCCTTGTAGACACCTTGGTC |
| TGFB2 | AGACCCCACATCTCCTGCTAATC | AATCAATGTAAAGAGGGCGAAGGC |
| CD59a | TCACTGGCGATCTGAAAAGTGTCTA | GCAGCACTATCTTGAGCCACATC |
| ABCA1 | GGTTTGGAGATGGTTATACAATAGTTGT | TTCCCGGAAACGCAAGTC |
| HPRT | TTGCTCGAGATGTCATGAAGGA | AGCAGGTCAGCAAAGAACTTATAG |
| RPL27 | TGAAAGGTTAGCGGAAGTGC | TTTCATGAACTTGCCCATCTC |

### Collagen Synthesis Assay

To measure collagen production by vSMC, cells were seeded at a density of 0.2*10⁶ cells per well. After attachment of the cells, control medium or medium containing GSO-control, GSO-494; -495 or -329 (5 ng/ml), was added. Subsequently, 1 μCi [³H]proline (Perkin Elmer) in the presence of 50 μg/mL ascorbic acid was added and incubated overnight at 37° C. Cells were detached from the wells in 20 mM Tris*HCl/0.36 mM CaCl₂ (pH=7.6) and sonicated for 2 minutes. Collagen was degraded by incubation with 100 U/mL collagenase for 2 hours at 37° C., after which samples were centrifuged for 15 minutes at 13.2 g. Proteins were precipitated for 30 minutes on ice using 50% trichloroacetic acid, after which [³H]proline content in the supernatant as a measure for collagen production was quantified in a liquid scintillation analyzer (Packard 1500 Tricarb, USA). Protein content was measured using a standard BCA protein assay.

### Statistical Analysis

Data are expressed as mean±SEM. Two-tailed Student's t-tests were used to compare individual groups in the in vivo studies. Non-parametric data were analyzed using a Mann-Whitney U test. A level of P<0.05 was considered significant.

### Results

### MiR-494, -495 and -329 are Predicted to Regulate Multiple Atherosclerosis-Related Genes

We performed a reversed target prediction based on a list we compiled of 150 atherosclerosis-related genes known from literature and previous studies. As expected, we identified multiple miRs that have previously been described in atherosclerosis, including miR-143/145,^{xxiii} miR-23/24^{xxiv} and miR-33.¹¹ Less established miRs were identified as well, such as miR-590-3p. Mir-590-3p was predicted to target the most genes, which is in line with the recent finding that miR-590-3p is regulated in minimally-oxidized LDL induced vSMC phenotype transformation.^{xxv}

Interestingly, we found enrichment of binding sites for multiple miRs from one single miR-gene cluster, located in an imprinted region on the long arm of chromosome 14 (14q32 (chromosome 12f1 in mice) (Table 1a). We identified 11 miRs of this gene cluster, including miR-329 (23 pro- and 11 anti-atherogenic targets), miR-494 (30 pro- and 8 anti-atherogenic targets) and miR-495 (35 pro- and 10 anti-atherogenic targets), which were selected for further investigations. The 14q32 miR gene cluster is highly conserved between mammals, and more specifically, between mice and men, and we found that most, but not all, putative target sites are conserved as well.

Target Gene Expression is Up-Regulated In Vitro after Inhibition of miRs by GSOs

In order to verify upregulation of target gene expression after inhibition of miR-494, -495 and -329, cultured cells were treated with GSOs. To reflect the involvement of various cell types in atherosclerosis, smooth muscle cells, endothelial cells, macrophages, fibroblasts and mast cells were used for these assays. Multiple target genes among which are cytokines, complement components, lipid-related targets and tissue inhibitors of metalloproteinases (TIMPs), were investigated in order to examine the broad effects miRs can exert.

Inhibition of miR-494 led to a significant up-regulation of the chemokine receptor CXCR4 in both endothelial cells and smooth muscle cells. Also, its ligand CXCL12 (SDF-1) was significantly increased in macrophages and mast cells. It has previously been shown that CXCR4/CXCL12 plays a protective role in atherosclerosis.^{xxvi,xxvii} Inhibition of miR-494 also led to an up-regulation of ACVR1 (a member of the TGF-beta superfamily) and of ADIPOR2 (involved in fatty acid oxidation) (FIG. 17). Inhibition of miR-495 led to a significant increase of CDKN1B expression in fibroblasts. CDKN1B (p27Kip1) is expressed in atherosclerotic plaques and has the ability to inhibit vSMC proliferation.^{xxvviii} The expression of the complement regulatory protein CD59 was also increased in macrophages, as well as the expression of the anti-inflammatory cytokine IL-10 (FIG. 18). Target gene expression after inhibition of miR-329 revealed an up-regulation of ADIPOR2, CD59, IL10RA and CXCL12 (FIG. 19).

Inhibition of miR-494, -495 and -329 Reduces Atherosclerotic Lesion Formation

Atherosclerotic lesions were induced in apoE^{-/-} mice by placement of perivascular collars around both carotid arteries. Four days after surgery mice received GSOs against miR-494, mir-495 or miR-329; control groups received either PBS or GSO-control. Downregulation of miR-494 (46%), miR-495 (23%) and miR-329 (35%) expression was detected in the carotid arteries at 3 days after GSO injection (FIG. 20).

To assess the effect of inhibiting miR-494, -495 and -329 on the development of atherosclerosis, mice were sacrificed four weeks after collar placement and plaques were analyzed for size and composition. HPS stained sections revealed a reduction of 65% in atherosclerotic plaque size in the group treated with GSO-494 (GSO-control: 47±11*10³ μm²; GSO-494: 16±3*10³ μm²; p<0.05; FIG. 12). Treatment with GSO-495 led to a decrease of 52% in lesion size (GSO-495: 22±5*10³ μm²; p<0.05 compared to GSO-control). Mice treated with GSO-329 also showed a reduction in plaque size (42%), which did not reach significance (GSO-329: 27±6*10³ μm²; p=0.13 compared to GSO-control). We did not observe differences in lesion size between PBS and control GSO treated groups (GSO-control: 47±11*10³ μm²; PBS: 38±11*10; p=0.47; FIG. 12), illustrating that the GSO-control did not exert aspecific effects.

Treatment with GSO-494 and -495 Leads to an Enhanced Stable Phenotype of Atherosclerotic Lesions

Interestingly, atherosclerotic plaques were not only reduced in size after treatment with GSOs; the plaques also showed an increase in plaque stability. So-called ‘stable lesions’ are characterized by a small necrotic core and a thick fibrous cap rich in collagen and smooth muscle cells. Indeed, necrotic core size was significantly reduced by 80% in mice treated with GSO-494 (control-GSO: 33±6%; GSO-494: 6±3%; P<0.001; FIG. 13) and by 60% in mice treated with GSO-495 (control-GSO: 33±6%; GSO-495: 13±5%; P<0.05). Furthermore, collagen content showed a significant increase after inhibition of miR-494 (control-GSO: 6.6±1.6%; GSO-494: 12.7±2.1%; P<0.05) and miR-495 (control-GSO: 6.6±1.6%; GSO-495: 15.8±3.1%; P<0.02). Taken together, the decrease in necrotic core size with concomitant increase in collagen content illustrates enhanced plaque stability.

Plaque morphometry was further examined by visualizing smooth muscle cells using an anti-alpha smooth muscle actin antibody. The percentage of positively stained lesion area was similar in the treatment groups (control-GSO: 4.6±1.0%; GSO-494: 6.4±1.8%; GSO-495: 4.7±1.6%; GSO-329: 6.0±1.3%). Macrophage content was only decreased in the group treated with GSO-495 (control-GSO: 20.0±2.2%; GSO-495: 14.1±1.3%; P<0.05). No differences were found in either mast cell numbers (control-GSO: 2.9±0.6 mast cells/mm²; GSO-494: 2.2±0.5 mast cells/mm²; GSO-495: 2.0±0.4 mast cells/mm²; GSO-329: 2.5±0.6 mast cells/mm²) or in their activation status (data not shown).

Reduction in Cholesterol Levels after Inhibition of miR-494 and -495

Total cholesterol levels showed a reduction after treatment with GSO-494 (control-GSO: 30.4±1.1 mM; GSO-494: 26.4±0.7 mM; P<0.01; FIG. 14A) and GSO-495 (GSO-495: 26.4±1.6; P=0.056 compared to control-GSO; FIG. 14A). Lipid profiling by AKTA-FPLC revealed a reduction in the VLDL fraction after treatment with GSO-494 and GSO-495 (FIG. 14B).

Inhibition of miR-329 and -495 Alter Numbers of Blood Lymphocytes and Neutrophils after GSO Treatment

Analysis of WBC by Sysmex cell differentiation analysis showed a decrease in absolute amount of lymphocytes after treatment with GSO-495 at time of sacrifice. Mice treated with GSO-329 also had reduced numbers of lymphocytes, as well as a decrease in the absolute amount of neutrophils in the blood. (FIG. 15)

Inhibition of miR-494 and -495 Results in Altered Collagen Homeostasis

In order to elucidate the mechanism behind the increased amount of collagen present in the plaque, we studied both of collagen synthesis and collagen degradation. We first looked at collagen synthesis in an in vitro setup. Inhibition of miR-494, -495 and -329 in smooth muscle cells did not result in increased collagen synthesis rate (FIG. 21).

Next we looked at collagen degradation. We determined TIMP expression after treatment of cultured cells with GSOs, as TIMPs inhibit collagen degradation by MMPs TIMP3 is a predicted target of miR-494 and miR-329, and TIMP2 is a target gene of miR-495. Indeed, the expression of TIMP3 was increased in endothelial cells after treatment with GSO-329. Also, expression levels of TIMP3 were increased in mast cells and macrophages after inhibition of miR-494. Inhibition of miR-495 led to up-regulation of TIMP2 expression in mast cells. We then measured expression of MMPs which are known to be important in plaque stability as they can degrade collagen, including MMP2, MMP8, MMP9 and MMP12. Only MMP8 is a predicted target of miR-495, and indeed we observed an increase of MMP8 expression after inhibition of miR-495. The expression levels of the other MMPs remained unchanged, resulting in a net increase in TIMP/MMP ratio (FIG. 16). Therefore, increased collagen content in the plaque is most likely caused by decreased degradation instead of increased synthesis of collagen.

### Discussion

The current study is the first to report a role for miR-494, miR-495 and miR-329, all members of the 14q32 miR gene cluster, in the development of atherosclerosis. We used a unique strategy to identify these miRs, by combining knowledge from our own previous experiments and literature in an in silico approach. For this Reversed Target Prediction, we compiled two lists of miRs, predicted to either aggravate or ameliorate atherosclerosis. As expected, many of the miRs identified by this approach have been described in literature to affect vascular inflammation. Besides recovering miRs known to be important in atherosclerosis, we were able to identify miRs that have not been investigated yet in this disease, in particular those from the 14q32 cluster. The 14q32 miR gene cluster is highly conserved in mammals and consists of 59 miR genes in mice and 54 in human.^{xxix} Previously it has been shown that many of the 14q32 miRs are implicated in human disease.^{xxx}. To establish their role in atherosclerosis, we inhibited miR-494, miR-495 and miR-329 in an in vivo model for atherosclerosis. We observed adecrease in atherosclerotic plaque formation, with a concomitant increase in plaque stability.

Even though miR-494 was predicted to target more pro- than anti-inflammatory target genes in our RTP, the in vivo effects of inhibiting miR-494 revealed a positive effect on atherosclerosis. We showed that miR-494 inhibition, and inhibition of miR-329 and miR-495 for that matter, leads to upregulation of both pro- and anti-atherosclerotic genes. A number of studies have targeted pro-atherogenic genes in order to reduce atherosclerosis, but our data suggest that up-regulating anti-atherogenic genes may be just as, or even more, promising when treating this complex disease.

Reduction of cholesterol levels by statins is still the most commonly used treatment of atherosclerosis. Lowering of LDL levels after statin treatment ranges from 20 to 60%, which results in a reduction of cardiovascular events of around 30%.^{xxxi} Inhibition of miR-494 and miR-495 reduced cholesterol levels by only 13%, mainly by a reduction in VLDL fractions. Although this reduction almost certainly contributes, we believe that it is unlikely that the major reduction in plaque size is solely due to this relatively modest decrease in plasma cholesterol. Furthermore, the observed change in plasma lipoproteins does not fully explain the prominent increase in plaque stability seen in our study, and we thus aimed to further elucidate the effects of miRNA inhibition on matrix homeostasis. Inhibition of miR-494 and miR-495 proved to be effective in improving atherosclerotic plaque stability as illustrated by a decrease in necrotic core size and an increase in collagen content. Since we detected no differences in smooth muscle cell content, we looked specifically at the increased amount of collagen and hypothesized that this may be caused by increased collagen homeostasis. No changes in collagen synthesis rate were detected after treating smooth muscle cells in vitro with GSOs. However, the TIMP/MMP ratio was significantly increased, indicative of decreased collagen degradation. In vivo, decreased collagen degradation will result in an increased thickness of the fibrous cap, thereby reducing the risk of plaque rupture with concomitant cardiovascular events.

Taken together, our results indicate that miRs are able to fine tune the expression of multiple genes rather than completely inhibit their target genes. Because miRs can regulate a large number of individual genes, delicate changes in gene expression may add up to a large net effect. In this study, inhibition of 14q32 miRs affects many atherosclerosis-related genes, resulting in a striking decrease in atherosclerotic lesion development and progression.

Involvement of the 14q32 miR gene cluster has not been previously shown in atherosclerosis; however, recently our group has described a role of these miRs in therapeutic neovascularization³⁸. For patients suffering from ischemia, such as in peripheral artery disease and after myocardial infarction, increasing blood flow to the tissues is crucial. Stimulating arterio- and angiogenesis is always accompanied by an inflammatory reaction, which often leads to an aggravation of the underlying cause of the ischemia: atherosclerosis. This so-called Janus phenomenon^{xxxii} is a major drawback in this field of research, especially in the clinic. Intriguingly, our group showed that inhibition of miR-494, -495 and -329 leads to a profound increase in blood flow after hind limb ischemia^{xxxiii}. Inhibiting the 14q32 gene cluster may therefore be unique in inducing angio- and arteriogenesis while simultaneously reducing atherosclerosis.

In conclusion, we discovered a role for the 14q32 miR gene cluster in atherosclerosis by utilizing the specific characteristic of miRs to regulate multiple genes and cellular processes. Inhibition of 14q32 miRs-494, -495 and -329 led to a reduction in lesion size, an increase in plaque stability and a reduction of cholesterol levels. This makes 14q32 miRs promising new therapeutic targets for patients suffering from cardiovascular disease.

### REFERENCES

(1) van Oostrom M C, van O O, Quax P H, Verhaar M C, Hoefer I E. Insights into mechanisms behind arteriogenesis: what does the future hold? J Leukoc Biol 2008; 84:1379-91.
(2) Risau W. Mechanisms of angiogenesis. Nature 1997; 386:671-4.
(3) Germani A, Di C C, Pompilio G, Biglioli P, Capogrossi M C. Regenerative therapy in peripheral artery disease. Cardiovasc Ther 2009; 27:289-304.
(4) Gupta R, Tongers J, Losordo D W. Human studies of angiogenic gene therapy. Circ Res 2009; 105:724-36.
(5) Bartel D P. MicroRNAs: genomics, biogenesis, mechanism, and function. Cell 2004; 116:281-97.
(6) van SC, Seghers L, Bijkerk R, Duijs J M, Roeten M K, van Oeveren-Rietdijk A M, Baelde H J, Monge M, Vos J B, de Boer H C, Quax P H, Rabelink T J, van Zonneveld A J. Antagomir-mediated silencing of endothelial cell specific microRNA-126 impairs ischemia-induced angiogenesis. J Cell Mol Med 2009; 13:1577-85.
(7) Grundmann S, Hans F P, Kinniry S, Heinke J, Helbing T, Bluhm F, Sluijter J P, Hoefer I, Pasterkamp G, Bode C, Moser M. MicroRNA-100 regulates neovascularization by suppression of mammalian target of rapamycin in endothelial and vascular smooth muscle cells. Circulation 2011; 123:999-1009.
(8) Hazarika S, Farber C R, Dokun A O, Pitsillides A N, Wang T, Lye R J, Annex B H. MicroRNA-93 Controls Perfusion Recovery Following Hind-Limb Ischemia by Modulating Expression of Multiple Genes in the Cell Cycle Pathway. Circulation 2013.
(9) Semo J, Sharir R, Afek A, Avivi C, Barshack I, Maysel-Auslender S, Krelin Y, Kain D, Entin-Meer M, Keren G, George J. The 106b˜25 microRNA cluster is essential for neovascularization after hindlimb ischaemia in mice. Eur Heart J 2013.
(10) Bonauer A, Carmona G, Iwasaki M, Mione M, Koyanagi M, Fischer A, Burchfield J, Fox H, Doebele C, Ohtani K, Chavakis E, Potente M, Tjwa M, Urbich C, Zeiher A M, Dimmeler S. MicroRNA-92a controls angiogenesis and functional recovery of ischemic tissues in mice. Science 2009; 324:1710-3.
(11) Yin K J, Olsen K, Hamblin M, Zhang J, Schwendeman S P, Chen Y E. Vascular endothelial cell-specific microRNA-15a inhibits angiogenesis in hindlimb ischemia. J Biol Chem 2012; 287:27055-64.
(12) Spinetti G, Fortunato O, Caporali A, Shantikumar S, Marchetti M, Meloni M, Descamps B, Floris I, Sangalli E, Vono R, Faglia E, Specchia C, Pintus G, Madeddu P, Emanueli C. MicroRNA-15a and microRNA-16 impair human circulating proangiogenic cell functions and are increased in the proangiogenic cells and serum of patients with critical limb ischemia. Circ Res 2013; 112:335-46.
(13) Caporali A, Emanueli C. MicroRNAs in Postischemic Vascular Repair. Cardiol Res Pract 2012; 2012:486702.
(14) Caporali A, Emanueli C. MicroRNA-503 and the extended microRNA-16 family in angiogenesis. Trends Cardiovasc Med 2011; 21:162-6.
(15) Seitz H, Royo H, Bortolin M L, Lin S P, Ferguson-Smith A C, Cavaille J. A large imprinted microRNA gene cluster at the mouse Dlk1-Gtl2 domain. Genome Res 2004; 14:1741-8.
(16) Benetatos L, Hatzimichael E, Londin E, Vartholomatos G, Loher P, Rigoutsos I, Briasoulis E. The microRNAs within the DLK1-DIO3 genomic region: involvement in disease pathogenesis. Cell Mol Life Sci 2013; 70:795-814.
(17) Gordon F E, Nutt C L, Cheunsuchon P, Nakayama Y, Provencher K A, Rice K A, Zhou Y, Zhang X, Klibanski A. Increased expression of angiogenic genes in the brains of mouse meg3-null embryos. Endocrinology 2010; 151:2443-52.
(18) Snyder C M, Rice A L, Estrella N L, Held A, Kandarian S C, Naya F J. MEF2A regulates the Gtl2-Dio3 microRNA mega-cluster to modulate WNT signaling in skeletal muscle regeneration. Development 2013; 140:31-42.
(19) Zhao W, Zhao S P, Peng D Q. The effects of myocyte enhancer factor 2A gene on the proliferation, migration and phenotype of vascular smooth muscle cells. Cell Biochem Funct 2012; 30:108-13.
(20) Firulli A B, Miano J M, Bi W, Johnson A D, Casscells W, Olson E N, Schwarz J J. Myocyte enhancer binding factor-2 expression and activity in vascular smooth muscle cells. Association with the activated phenotype. Circ Res 1996; 78:196-204.
(21) Bhagat L, Putta M R, Wang D, Yu D, Lan T, Jiang W, Sun Z, Wang H, Tang J X, La M N, Kandimalla E R, Agrawal S. Novel oligonucleotides containing two 3′-ends complementary to target mRNA show optimal gene-silencing activity. J Med Chem 2011; 54:3027-36.
(22) Hellingman A A, Bastiaansen A J, de Vries M R, Seghers L, Lijkwan M A, Lowik C W, Hamming J F, Quax P H. Variations in surgical procedures for hind limb ischaemia mouse models result in differences in collateral formation. Eur J Vasc Endovasc Surg 2010; 40:796-803.
(23) Ghosh G, Subramanian I V, Adhikari N, Zhang X, Joshi H P, Basi D, Chandrashekhar Y S, Hall J L, Roy S, Zeng Y, Ramakrishnan S. Hypoxia-induced microRNA-424 expression in human endothelial cells regulates HIF-alpha isoforms and promotes angiogenesis. J Clin Invest 2010; 120:4141-54.0
(24) Nossent A Y, Eskildsen T, Andersen L B, Bie P, Bronnum H, Schneider M, Andersen D, Welten S M, Jeppesen P L, Hamming I F, Hansen J L, Quax P H, Sheikh S P. The 14q32 MicroRNA-487b Targets the Anti-Apoptotic Insulin Receptor Substrate 1 in Hypertension Induced Remodeling of the Aorta. Ann Surg 2013; In Press.
(25) Semo J, Sharir R, Afek A, Avivi C, Barshack I, Maysel-Auslender S, Krelin Y, Kain D, Entin-Meer M, Keren G, George J. The 106b˜25 microRNA cluster is essential for neovascularization after hindlimb ischaemia in mice. Eur Heart J2013.
(26) Chamorro-Jorganes A, Araldi E, Penalva L O, Sandhu D, Fernandez-Hernando C, Suarez Y. MicroRNA-16 and microRNA-424 regulate cell-autonomous angiogenic functions in endothelial cells via targeting vascular endothelial growth factor receptor-2 and fibroblast growth factor receptor-1. Arterioscler Thromb Vasc Blot 2011; 31:2595-606.
(27) Nossent A Y, Hansen J L, Doggen C, Quax P H, Sheikh S P, Rosendaal F R. SNPs in microRNA binding sites in 3′-UTRs of RAAS genes influence arterial blood pressure and risk of myocardial infarction. Am J Hypertens 2011; 24:999-1006.
(28) Xi S, Xu H, Shan J, Tao Y, Hong J A, Inchauste S, Zhang M, Kunst T F, Mercedes L, Schrump D S. Cigarette smoke mediates epigenetic repression of miR-487b during pulmonary carcinogenesis. J Clin Invest 2013; 123:1241-61.
(29) Hwang-Verslues W W, Chang P H, Wei P C, Yang C Y, Huang C K, Kuo W H, Shew J Y, Chang K J, Lee E Y, Lee W H. miR-495 is upregulated by E12/E47 in breast cancer stem cells, and promotes oncogenesis and hypoxia resistance via downregulation of E-cadherin and REDD1. Oncogene 2011; 30:2463-74.
(30) Jiang X, Huang H, Li Z, He C, Li Y, Chen P, Gurbuxani S, Arnovitz S, Hong G M, Price C, Ren H, Kunjamma R B, Neilly M B, Salat J, Wunderlich M, Slany R K, Zhang Y, Larson R A, Le Beau M M, Mulloy J C, Rowley J D, Chen J. MiR-495 is a tumor-suppressor microRNA down-regulated in MLL-rearranged leukemia. Proc Natl Acad Sci USA 2012; 109:19397-402.
(31) Li Z, Cao Y, Jie Z, Liu Y, Li Y, Li J, Zhu G, Liu Z, Tu Y, Peng G, Lee D W, Park S S. miR-495 and miR-551a inhibit the migration and invasion of human gastric cancer cells by directly interacting with PRL-3. Cancer Lett 2012; 323:41-7.
(32) Wang X, Zhang X, Ren X P, Chen J, Liu H, Yang J, Medvedovic M, Hu Z, Fan G C. MicroRNA-494 targeting both proapoptotic and antiapoptotic proteins protects against ischemia/reperfusion-induced cardiac injury. Circulation 2010; 122:1308-18.
(33) Ohdaira H, Sekiguchi M, Miyata K, Yoshida K. MicroRNA-494 suppresses cell proliferation and induces senescence in A549 lung cancer cells. Cell Prolif 2012; 45:32-8.
ⁱ Galkina E, Ley K. Immune and inflammatory mechanisms of atherosclerosis. Annu Rev Immunol. 2009; 27:165-97.
ⁱⁱ Libby P. Inflammation in atherosclerosis. Arterioscler Thromb Vasc Biol. 2012 September; 32(9):2045-51.
ⁱⁱⁱ Keaney J F Jr. Immune modulation of atherosclerosis. Circulation. 2011 Nov. 29; 124(22):e559-60.
^{iv} Virmani R, Kolodgie F D, Burke A P, Farb A, Schwartz S M. Lessons from sudden coronary death: a comprehensive morphological classification scheme for atherosclerotic lesions. Arterioscler Thromb Vasc Biol. 2000 May; 20(5):1262-75.
^{v} Shah P K. Mechanisms of plaque vulnerability and rupture. J Am Coll Cardiol. 2003 Feb. 19; 41(4 Suppl S): 15S-22S.
^{vi} Chen K, Rajewsky N. The evolution of gene regulation by transcription factors and microRNAs. Nat Rev Genet. 2007 February; 8(2):93-103
^{vii} Rajewsky N. MicroRNA target predictions in animals. Nat Genet. 2006 June; 38 Suppl: S8-13.
^{viii} Rayner K J, Esau C C, Hussain F N, McDaniel A L, Marshall S M, van Gils J M, Ray T D, Sheedy F J, Goedeke L, Liu X, Khatsenko O G, Kaimal V, Lees C J, Fernandez-Hernando C, Fisher E A, Temel R E, Moore K J. Inhibition of miR-33a/b in non-human primates raises plasma HDL and lowers VLDL triglycerides. Nature. 2011 Oct. 19; 478(7369):404-7.
^{ix} Sun C, Alkhoury K, Wang Y I, Foster G A, Radecke C E, Tam K, Edwards C M, Facciotti M T, Armstrong E J, Knowlton A A, Newman J W, Passerini A G, Simon S I. IRF-1 and miRNA126 modulate VCAM-1 expression in response to a high-fat meal. Circ Res. 2012 Sep. 28; 111(8): 1054-64
^{x} Fang Y, Davies P F. Site-specific microRNA-92a regulation of Kruppel-like factors 4 and 2 in atherosusceptible endothelium. Arterioscler Thromb Vasc Biol. 2012 April; 32(4):979-87.
^{xi} Alaiti M A, Orasanu G, Tugal D, Lu Y, Jain M K. Kruppel-like factors and vascular inflammation: implications for atherosclerosis. Curr Atheroscler Rep. 2012 October; 14(5):438-49
^{xii} Wang Y S, Wang H Y, Liao Y C, Tsai P C, Chen K C, Cheng H Y, Lin R T, Juo S H. MicroRNA-195 regulates vascular smooth muscle cell phenotype and prevents neointimal formation. Cardiovasc Res. 2012 Sep. 1; 95(4):517-26.
^{xiii} Nazari-Jahantigh M, Wei Y, Noels H, Akhtar S, Zhou Z, Koenen R R, Heyll K, Gremse F, Kiessling F, Grommes J, Weber C, Schober A. MicroRNA-155 promotes atherosclerosis by repressing Bcl6 in macrophages. J Clin Invest. 2012 Nov. 1; 122(11):4190-202
^{xiv} Seo D, Wang T, Dressman H, Herderick E E, Iversen E S, Dong C, Vata K, Milano C A, Rigat F, Pittman J, Nevins J R, West M, Goldschmidt-Clermont P J. Gene expression phenotypes of atherosclerosis. Arterioscler Thromb Vasc Biol. 2004 October; 24(10):1922-7.
^{xv} Tabibiazar R, Wagner R A, Ashley E A, King J Y, Ferrara R, Spin J M, Sanan D A, Narasimhan B, Tibshirani R, Tsao P S, Efron B, Quertermous T. Signature patterns of gene expression in mouse atherosclerosis and their correlation to human coronary disease. Physiol Genomics. 2005 Jul. 14; 22(2):213-26.
Fu S, Zhao H, Shi J, Abzhanov A, Crawford K, Ohno-Machado L, Zhou J, Du Y, Kuo W P, Zhang J, Jiang M, Jin J G. Peripheral arterial occlusive disease: global gene expression analyses suggest a major role for immune and inflammatory responses. BMC Genomics. 2008 Aug. 1; 9:369.
^{xvii} Laukkanen J, Ylä-Herttuala S. Genes involved in atherosclerosis. Exp Nephrol. 2002; 10(2): 150-63.
^{xxviii} Lusis A J. Genetics of atherosclerosis. Trends Genet. 2012 June; 28(6):267-75.
^{xix} Von der Thüsen J H, van Berkel T J C, Biessen E A L. Induction of rapid atherogenesis by perivascular carotid collar placement in apolipoprotein E-deficient and low-density lipoprotein receptor-deficient mice. Circulation. 2001; 103:1164-1170.
^{xx} Zhao Y, Pennings M, Hildebrand R B, Ye D, Calpe-Berdiel L, Out R, Kjerrulf M, Hurt-Camejo E, Groen A K, Hoekstra M, Jessup W, Chimini G, Van Berkel T J, Van Eck M. Enhanced foam cell formation, atherosclerotic lesion development, and inflammation by combined deletion of ABCA1 and SR-BI in Bone marrow-derived cells in LDL receptor knockout mice on western-type diet Circ Res. 2010 Dec. 10; 107(12):e20-31.
^{xxi} Razin E, Marx G. Thrombin-induced degranulation of cultured bone marrow-derived mast cells. J Immunol 1984; 133:3282-3285.
^{xxii} McGookin R. RNA extraction by the guanidine thiocyanate procedure. Methods Mol Biol. 1985; 2:113-6.
^{xxiii} Rader D J, Parmacek M S. Secreted miRNAs suppress atherogenesis. Nat Cell Biol. 2012 Feb. 29; 14(3):233-5.
^{xxiv} Boon R A, Hergenreider E, Dimmeler S. Atheroprotective mechanisms of shear stress-regulated microRNAs. Thromb Haemost. 2012 October; 108(4):616-20.
^{xxv} Karagiannis G S, Weile J, Bader G D, Minta J. Integrative pathway dissection of molecular mechanisms of moxLDL-induced vascular smooth muscle phenotype transformation. BMC Cardiovasc Disord. 2013 Jan. 16; 13:4
^{xxvi} Zernecke A, Bot I, Djalali-Talab Y, Shagdarsuren E, Bidzhekov K, Meiler S, Krohn R, Schober A, Sperandio M, Soehnlein O, Bornemann J, Tacke F, Biessen E A, Weber C. Protective role of CXC receptor 4/CXC ligand 12 unveils the importance of neutrophils in atherosclerosis. Circ Res. 2008 Feb. 1; 102(2):209-17.
^{xxvii} Akhtar S, Gremse F, Kiessling F, Weber C, Schober A. CXCL12 Promotes the Stabilization of Atherosclerotic Lesions Mediated by Smooth Muscle Progenitor Cells in Apoe-Deficient Mice. Arterioscler Thromb Vasc Biol. 2013 April; 33(4):679-86.
^{xxviii} Shahzad K, Thati M, Wang H, Kashif M, Wolter J, Ranjan S, He T, Zhou Q, Blessing E, Bierhaus A, Nawroth P P, Isermann B. Minocycline reduces plaque size in diet induced atherosclerosis via p27(Kip1). Atherosclerosis. 2011 November; 219(1):74-83.
^{xxix} Seitz H, Royo H, Bortolin M L, Lin S P, Ferguson-Smith A C, Cavaille J. A large imprinted microRNA gene cluster at the mouse Dlk1-Gtl2 domain. Genome Res 2004; 14:1741-8.
^{xxx} Benetatos L, Hatzimichael E, Londin E, Vartholomatos G, Loher P, Rigoutsos I, Briasoulis E. The microRNAs within the DLK1-DIO3 genomic region: involvement in disease pathogenesis. Cell Mol Life Sci 2013; 70:795-814.
^{xxxi} Law M R, Wald N J, Rudnicka A R. Quantifying effect of statins on low density lipoprotein cholesterol, ischaemic heart disease, and stroke: systematic review and meta-analysis. BMJ. 2003 Jun. 28; 326(7404):1423.
^{xxxii} Epstein S E, Stabile E, Kinnaird T, Lee C W, Clavijo L, Burnett M S. Janus phenomenon: the interrelated tradeoffs inherent in therapies designed to enhance collateral formation and those designed to inhibit atherogenesis. Circulation. 2004 Jun. 15; 109(23):2826-31.
^{xxxiii} Bastiaansen T, Welten S. J. M, et al. Inhibition of 14q32 MicroRNAs Drastically Improves Blood Flow Recovery After Ischemia. Circulation 2012; 126: A16334.

1. A method for modulating vascular re-modelling processes and/or for treating or preventing a vascular disorder, said method comprising administering to a subject in need thereof a modulator of one or more 14q32 microRNAs.

2. The method of claim 1, wherein the vascular disorder is a disorder, disease, syndrome and/or condition affecting any of the vessels and/or vascular systems of the human or animal body.

3. The method of claim 1, wherein the modulator is a modulator of 14q32 microRNA expression.

4. The method of claim 1, wherein the modulator is a molecule or compound capable of increasing or inhibiting/decreasing the expression of a 14q32 microRNA.

5. The method of claim 1, wherein the modulator is an inhibitor of one or more of the 14q32 microRNAs.

6. The method of claim 1, wherein the the vascular disorder is peripheral artery disease and/or the modulation of vascular re-modelling processes is vascular re-modelling processes in peripheral arteries.

7. The method of claim 1, wherein the modulation of vascular re-modelling processes is in cardiovascular/coronary and/or cerebral vessels.

8. The method of claim 1, wherein the vascular disorder is cardiovascular/coronary and/or cerebral artery diseases.

9. The method of claim 1, wherein the vascular disorder is restenosis and/or atherosclerosis.

10. The method of claim 1, wherein the modulation of vascular remodelling processes is vascular remodelling processes which occur following mycocardial infarction and the vascular disorder is aneurysm formation.

11. The method of claim 1, wherein the vascular disorder is restenosis as might occur following, surgical procedures or interventions.

12. The method of claim 1, wherein the vascular disorder is hypercholesterolemia.

13. A method for for modulating plaque stability and/or for treating or preventing restenosis comprising administering a modulator of one or more 14q32 microRNAs.

14. The method of claim 1, wherein the modulators are modulators of one or more microRNAs selected from the group consisting of:
1) microRNA-2392
2) microRNA-770
3) microRNA-493
4) microRNA-337
5) microRNA-665
6) microRNA-431
7) microRNA-433
8) microRNA-127
9) microRNA-432
10) microRNA-136
11) microRNA-370
12) microRNA-379
13) microRNA-411
14) microRNA-299
15) microRNA-380
16) microRNA-1197
17) microRNA-323a
18) microRNA-758
19) microRNA-329-1
20) microRNA-329-2
21) microRNA-494
22) microRNA-1193
23) microRNA-543
24) microRNA-495
25) microRNA-376c
26) microRNA-376a-2
27) microRNA-654
28) microRNA-376b
29) microRNA-376a-1
30) microRNA-300
31) microRNA-1185-1
32) microRNA-1185-2
33) microRNA-381
34) microRNA-487b
35) microRNA-539
36) microRNA-889
37) microRNA-544a
38) microRNA-655
39) microRNA-487a
40) microRNA-382
41) microRNA-134
42) microRNA-668
43) microRNA-485
44) microRNA-323b
45) microRNA-154
46) microRNA-496
47) microRNA-377
48) microRNA-541
49) microRNA-409
50) microRNA-412
51) microRNA-369
52) microRNA-410
53) microRNA-656
54) microRNA-1247.

15. The method of claim 14, wherein the modulators are modulators of one or more of miR-329, miR-494, miR-487b and/or miR-495.

16. The method of claim 1, wherein the modulators are DNA and/or RNA based antisense oligonucleotides.

17. (canceled)

18. (canceled)

19. The method of claim 13, wherein the modulator is a modulator of one or more microRNAs selected from the group consisting of:
1) microRNA-2392
2) microRNA-770
3) microRNA-493
4) microRNA-337
5) microRNA-665
6) microRNA-431
7) microRNA-433
8) microRNA-127
9) microRNA-432
10) microRNA-136
11) microRNA-370
12) microRNA-379
13) microRNA-411
14) microRNA-299
15) microRNA-380
16) microRNA-1197
17) microRNA-323a
18) microRNA-758
19) microRNA-329-1
20) microRNA-329-2
21) microRNA-494
22) microRNA-1193
23) microRNA-543
24) microRNA-495
25) microRNA-376c
26) microRNA-376a-2
27) microRNA-654
28) microRNA-376b
29) microRNA-376a-1
30) microRNA-300
31) microRNA-1185-1
32) microRNA-1185-2
33) microRNA-381
34) microRNA-487b
35) microRNA-539
36) microRNA-889
37) microRNA-544a
38) microRNA-655
39) microRNA-487a
40) microRNA-382
41) microRNA-134
42) microRNA-668
43) microRNA-485
44) microRNA-323b
45) microRNA-154
46) microRNA-496
47) microRNA-377
48) microRNA-541
49) microRNA-409
50) microRNA-412
51) microRNA-369
52) microRNA-410
53) microRNA-656
54) microRNA-1247.

20. The method of claim 19, wherein the modulators are modulators of one or more of miR-329, miR-494, miR-487b and/or miR-495.

21. The method of claim 13, wherein the modulators the modulators are DNA and/or RNA based antisense oligonucleotides.


SEQUENCE LISTING (52 sequences)

SEQ ID NO: 1 - RNA, 22 nt, Homo sapiens
aaucguacagggucauccacuu

SEQ ID NO: 2 - RNA, 22 nt, Homo sapiens
ugaaacauacacgggaaaccuc

SEQ ID NO: 3 - RNA, 22 nt, Homo sapiens
aaacaaacauggugcacuucuu

SEQ ID NO: 4 - RNA, 22 nt, Mus musculus
aacacacccagcuaaccuuuuu

SEQ ID NO: 5 - RNA, 22 nt, Homo sapiens
aacacaccugguuaaccucuuu

SEQ ID NO: 6 - DNA, 22 bp, Artificial Sequence
  Description of Artificial Sequence: Synthetic gene silencing oligonucleotide
aagtggatgaccctgtacgatt

SEQ ID NO: 7 - DNA, 22 bp, Artificial Sequence
  Description of Artificial Sequence: Synthetic gene silencing oligonucleotide
gaggtttcccgtgtatgtttca

SEQ ID NO: 8 - DNA, 22 bp, Artificial Sequence
  Description of Artificial Sequence: Synthetic gene silencing oligonucleotide
aagaagtgcaccatgtttgttt

SEQ ID NO: 9 - DNA, 22 bp, Artificial Sequence
  Description of Artificial Sequence: Synthetic gene silencing oligonucleotide
aaaaaggttagctgggtgtgtt

SEQ ID NO: 10 - DNA, 22 bp, Artificial Sequence
  Description of Artificial Sequence: Synthetic gene silencing oligonucleotide
aaagaggttaaccaggtgtgtt

SEQ ID NO: 11 - DNA, 19 bp, Artificial Sequence
  Description of Artificial Sequence: Synthetic negative control GSO
tggcaatacctcagcatgt

SEQ ID NO: 12 - RNA, 22 nt, Artificial Sequence
  Description of Artificial Sequence: Synthetic AntagomiR oligonucleotide
  MISC_FEATURE (1)..(3): Phosphorothioate linkages between nucleotides
  MISC_FEATURE (19)..(22): Phosphorothioate linkages between nucleotides
  MISC_FEATURE (22)..(22): Linked to a cholesterol group through a hydroxyprolinol-linkage
aaguggaugacccuguacgauu

SEQ ID NO: 13 - RNA, 22 nt, Artificial Sequence
  Description of Artificial Sequence: Synthetic AntagomiR oligonucleotide
  MISC_FEATURE (1)..(3): Phosphorothioate linkages between nucleotides
  MISC_FEATURE (19)..(22): Phosphorothioate linkages between nucleotides
  MISC_FEATURE (22)..(22): Linked to a cholesterol group through a hydroxyprolinol-linkage
gagguuucccguguauguuuca

SEQ ID NO: 14 - RNA, 22 nt, Artificial Sequence
  Description of Artificial Sequence: Synthetic AntagomiR oligonucleotide
  MISC_FEATURE (1)..(3): Phosphorothioate linkages between nucleotides
  MISC_FEATURE (19)..(22): Phosphorothioate linkages between nucleotides
  MISC_FEATURE (22)..(22): Linked to a cholesterol group through a hydroxyprolinol-linkage
aagaagugcaccauguuuguuu

SEQ ID NO: 15 - RNA, 22 nt, Artificial Sequence
  Description of Artificial Sequence: Synthetic AntagomiR oligonucleotide
  MISC_FEATURE (1)..(3): Phosphorothioate linkages between nucleotides
  MISC_FEATURE (19)..(22): Phosphorothioate linkages between nucleotides
  MISC_FEATURE (22)..(22): Linked to a cholesterol group through a hydroxyprolinol-linkage
aaaaagguuagcuggguguguu

SEQ ID NO: 16 - RNA, 22 nt, Artificial Sequence
  Description of Artificial Sequence: Synthetic AntagomiR oligonucleotide
  MISC_FEATURE (1)..(3): Phosphorothioate linkages between nucleotides
  MISC_FEATURE (19)..(22): Phosphorothioate linkages between nucleotides
  MISC_FEATURE (22)..(22): Linked to a cholesterol group through a hydroxyprolinol-linkage
aaagagguuaaccagguguguu

SEQ ID NO: 17 - RNA, 21 nt, Artificial Sequence
  Description of Artificial Sequence: Synthetic negative control AntagomiR oligonucleotide
  MISC_FEATURE (1)..(3): Phosphorothioate linkages between nucleotides
  MISC_FEATURE (18)..(21): Phosphorothioate linkages between nucleotides
  MISC_FEATURE (21)..(21): Linked to a cholesterol group through a hydroxyprolinol-linkage
augacuaucgcuauucgcaug

SEQ ID NO: 18-26 - skipped (9 entries)

SEQ ID NO: 27 - DNA, 19 bp, Artificial Sequence
  Description of Artificial Sequence: Synthetic forward primer
gtgatcctggtcatgggtt

SEQ ID NO: 28 - DNA, 23 bp, Artificial Sequence
  Description of Artificial Sequence: Synthetic forward primer
cggctgggttagcggagcagtgt

SEQ ID NO: 29 - DNA, 23 bp, Artificial Sequence
  Description of Artificial Sequence: Synthetic forward primer
gtttatctacacggccccctctt

SEQ ID NO: 30 - DNA, 23 bp, Artificial Sequence
  Description of Artificial Sequence: Synthetic forward primer
actgtgcaactttgtggagaggt

SEQ ID NO: 31 - DNA, 21 bp, Artificial Sequence
  Description of Artificial Sequence: Synthetic forward primer
tgcatcagtgacggtaaacca

SEQ ID NO: 32 - DNA, 23 bp, Artificial Sequence
  Description of Artificial Sequence: Synthetic forward primer
catgtttgccacccctcagtatc

SEQ ID NO: 33 - DNA, 21 bp, Artificial Sequence
  Description of Artificial Sequence: Synthetic forward primer
gacggcatcatctatgggaca

SEQ ID NO: 34 - DNA, 22 bp, Artificial Sequence
  Description of Artificial Sequence: Synthetic forward primer
gggtgagaagctgaagaccctc

SEQ ID NO: 35 - DNA, 23 bp, Artificial Sequence
  Description of Artificial Sequence: Synthetic forward primer
agaccccacatctcctgctaatc

SEQ ID NO: 36 - DNA, 25 bp, Artificial Sequence
  Description of Artificial Sequence: Synthetic forward primer
tcactggcgatctgaaaagtgtcta

SEQ ID NO: 37 - DNA, 28 bp, Artificial Sequence
  Description of Artificial Sequence: Synthetic forward primer
ggtttggagatggttatacaatagttgt

SEQ ID NO: 38 - DNA, 22 bp, Artificial Sequence
  Description of Artificial Sequence: Synthetic forward primer
ttgctcgagatgtcatgaagga

SEQ ID NO: 39 - DNA, 20 bp, Artificial Sequence
  Description of Artificial Sequence: Synthetic forward primer
tgaaaggttagcggaagtgc

SEQ ID NO: 40 - DNA, 18 bp, Artificial Sequence
  Description of Artificial Sequence: Synthetic reversed primer
tgacaggtgcagccggta

SEQ ID NO: 41 - DNA, 24 bp, Artificial Sequence
  Description of Artificial Sequence: Synthetic reversed primer
ccagcgttcggggaaccgtctgaa

SEQ ID NO: 42 - DNA, 23 bp, Artificial Sequence
  Description of Artificial Sequence: Synthetic reversed primer
atcttgccatctccttctgcctt

SEQ ID NO: 43 - DNA, 23 bp, Artificial Sequence
  Description of Artificial Sequence: Synthetic reversed primer
gagacactcattcttggaggtca

SEQ ID NO: 44 - DNA, 18 bp, Artificial Sequence
  Description of Artificial Sequence: Synthetic reversed primer
ggctctcgaagaaccggc

SEQ ID NO: 45 - DNA, 21 bp, Artificial Sequence
  Description of Artificial Sequence: Synthetic reversed primer
agccagcctatctgccctatg

SEQ ID NO: 46 - DNA, 23 bp, Artificial Sequence
  Description of Artificial Sequence: Synthetic reversed primer
gacttgttcgtactcatcccctg

SEQ ID NO: 47 - DNA, 22 bp, Artificial Sequence
  Description of Artificial Sequence: Synthetic reversed primer
tggccttgtagacaccttggtc

SEQ ID NO: 48 - DNA, 24 bp, Artificial Sequence
  Description of Artificial Sequence: Synthetic reversed primer
aatcaatgtaaagagggcgaaggc

SEQ ID NO: 49 - DNA, 23 bp, Artificial Sequence
  Description of Artificial Sequence: Synthetic reversed primer
gcagcactatcttgagccacatc

SEQ ID NO: 50 - DNA, 18 bp, Artificial Sequence
  Description of Artificial Sequence: Synthetic reversed primer
ttcccggaaacgcaagtc

SEQ ID NO: 51 - DNA, 24 bp, Artificial Sequence
  Description of Artificial Sequence: Synthetic reversed primer
agcaggtcagcaaagaacttatag

SEQ ID NO: 52 - DNA, 21 bp, Artificial Sequence
  Description of Artificial Sequence: Synthetic reversed primer
tttcatgaacttgcccatctc